(19) United States
(12) Patent Application Publication
(10) Pub. No.: US 2026/0226096 A1
(43) Pub. Date: Aug. 6, 2026

(54) MODIFIED PROTEINS OR PEPTIDES FOR COVALENT TARGETING

(71) Applicant: YEDA RESEARCH AND DEVELOPMENT CO. LTD., Rehovot (IL)

(72) Inventors: Nir LONDON, Rehovot (IL); Ronen GABIZON, Rehovot (IL); Barr TIVON, Rehovot (IL); Rambabu REDDI, Rehovot (IL)

(73) Assignee: YEDA RESEARCH AND DEVELOPMENT CO. LTD., Rehovot (IL)

(21) Appl. No.: 19/153,980

(22) PCT Filed: Feb. 20, 2024
(86) PCT No.: PCT/IL2024/050189

§ 371 Date: Aug. 6, 2025

Related U.S. Application Data
(60) Provisional application No. 63/520,959, filed on Aug. 22, 2023.

(30) Foreign Application Priority Data
Feb. 20, 2023 (IL) 300815

(51) Int. Cl. C07K 1/107; C07K 1/113; C07K 7/06

(52) U.S. Cl. CPC C07K 1/1077; C07K 1/1133; C07K 7/06

(57) ABSTRACT

This invention is directed to modified proteins or modified peptides (modified by an electrophile, such as acrylamide or acrylate) for covalent binding with target proteins.

### SEQUENCE LISTING

The instant application contains a Sequence Listing which has been submitted electronically in XML file format and is hereby incorporated by reference in its entirety. Said XML copy, created on Feb. 19, 2024, is entitled “P-623495-PC_ST26.xml”, and is 56,897 bytes in size.

### FIELD OF THE INVENTION

This invention is directed to modified proteins or modified peptides (modified by an electrophile, such as acrylamide or acrylate) for covalent binding with target proteins.

### BACKGROUND OF THE INVENTION

Covalent tool compounds and chemical probes have been established as powerful technology with diverse applications in chemical biology. These applications range from inhibitors used for therapeutic applications to covalent probes used to study the function and properties of target proteins. Covalent compounds have been developed targeting a large variety of proteins including kinases, G-protein coupled receptors, hydrolases and others, have found important uses as probes for proteomics and microscopy, and are also used in emerging applications such as targeted degradation. The advantages of covalent compounds in chemical biology stem from several aspects. The irreversible binding to the target achieves prolonged potent inhibition with short systemic exposure. Covalent binding to the target facilitates downstream processes involving denaturation and proteolysis of the target without loss of the bound probe, making them especially useful in proteomics. Lastly, covalent binders frequently show enhanced selectivity by targeting non-conserved nucleophilic residues. This is exemplified by the recently approved Sotorasib and Adagrasib, which selectively target Kras^{G12C}.

A surge in the development of novel warheads for small molecules has expanded the targetable scope of amino acids to include lysine, tyrosine, acidic residues, histidine and others. These chemistries have greatly expanded the spectrum of protein targets. However, the synthetic installation of a reactive group on a peptide or full protein is not trivial. The large number of nucleophilic amino acids on a protein or the relatively harsh deprotection conditions required for solid phase peptide synthesis complicates electrophile installation. Various approaches have emerged recently to functionalize native sequences and prepare protein-based covalent binders. These include genetic code expansion to incorporate reactive groups into protein sequences via unnatural amino acids, approaches utilizing enzymatic activation of proteins and site-selective chemical approaches. However, these techniques remain technically challenging, and there remains a need for simple approaches to expand the scope of targetable residues with high selectivity.

Despite the surge in research into covalent compounds, targets such as transcription factors and protein-protein interaction interfaces are difficult to target with small molecules due to their broad and shallow binding surfaces. The use of peptides or peptidomimetics has emerged as a powerful approach to address these issues. Peptide binders can cover a large surface area, can bind protein targets with high affinity and can frequently be derived from known protein-protein interactions.

Potent covalent peptide binders have been developed for targets such as the bacterial divisome, E3 ubiquitin ligases, the anti-apoptotic protein BFL-1, and others. Due to the size of peptides and their conformational flexibility, computational modeling can aid in the design and placement of the electrophile.

### SUMMARY OF THE INVENTION

In some embodiments, provided herein a modified protein or a modified peptide comprising a recombinant protein or a synthetic peptide modified by an electrophile, wherein the electrophile is covalently bound to a thiol group of a cysteine within the protein or peptide; wherein the electrophile is represented by the structure of Formula I;

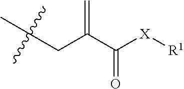

wherein,
   R¹ comprises substituted or unsubstituted alkyl, alkenyl, alkynyl, carbocycle, aryl, heteroaryl or heterocyclic;
   X comprises O, NR³ or substituted or unsubstituted alkylene; and R³ is hydrogen, alkyl, alkenyl, alkynyl, carbocycle, aryl, heteroaryl or heterocyclic;
wherein the modified protein or modified peptide is capable of covalently binding a target protein.

In some embodiments, the modified peptide comprises the following peptides: Ac-RSApSmCPSL-NH₂ (SEQ ID 3), Ac-RAHpSmCPASLQ-NH₂ (SEQ ID 8) or Ac-RAHpSSPASmCQ-NH₂ (SEQ ID 11); wherein pS is Phosphoserine and mC is Methacrylate-modified cysteine. In other embodiments, the modified peptide of Ac-RSApSmCPSL-NH₂(SEQ ID 3), Ac-RAHpSmCPASLQ-NH₂ (SEQ ID 8) or Ac-RAHpSSPASmCQ-NH₂ (SEQ ID 11); wherein pS is Phosphoserine and mC is Methacrylate-modified cysteine-covalently binds 14-3-3 target protein. In other embodiments, the peptide of Ac-RSApSmCPSL-NH₂ (SEQ ID 3), Ac-RAHpSmCPASLQ-NH₂ (SEQ ID 8) or Ac-RAHpSSPASmCQ-NH₂ (SEQ ID 11); covalently binds Cys38, Lys 122 or Lys 49 of 14-3-3σ target protein.

In some embodiments, the modified protein comprises a modified immunity (Im9) protein (SEQ ID 27). In other embodiments, the Im9 (SEQ ID 27) binds an E9 target protein.

In some embodiments, provided herein a method of preparing a modified protein or a modified peptide provided herein, wherein the method comprises;
a. identifying a target protein;
b. designing a peptide or protein covalent binder candidate based on previously characterized noncovalent binders of the target protein;
c. synthesizing a modified peptide or a modified protein, to allow recognition with the target protein at the recognition site; wherein said modified protein or modified peptide comprise a cysteine which is reacted with an electrophile of Formula I-IV.

In some embodiments, provided herein a protein-protein covalent conjugate comprising a modified protein described herein, covalently bound to a target protein, wherein the modified protein and the target protein possess non-covalent recognition prior to the covalent binding with the target protein.

In some embodiments, provided herein a peptide-protein covalent conjugate comprising a modified peptide described herein, covalently bound to a target protein, wherein the modified peptide and the target protein possess non-covalent recognition prior to the covalent binding with the target protein.

In some embodiment, provided herein a modified protein or modified peptide provided herein for use in selectively label, fluorescent label, inhibition, drug conjugation or conjugation to a target protein.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. Scheme for generating thioether-based modified protein or modified peptide of this invention. A cysteine residue can be introduced into peptides (ribbon-like line) or recombinant proteins (marked with *), which are then modified using an ethyl 2-(bromomethyl)acrylate. The resulting modified protein or modified peptide reacts with lysine or cysteine side chains proximal to the binding site on the receptor, to yield covalent adducts.

FIGS. 2A-2C. Generating peptide covalent reagents for 14-3-3 proteins. FIG. 2A: Structure of the complex of 14-3-3σ with phosphorylated peptide from YAP (; PDB: 3MHR). The non conserved cysteine 38 and the conserved lysines 49 and 122 are highlighted. FIG. 2B: Scheme for synthesis of electrophilic peptides. a) 20% piperidine in DMF, 3×4 minutes; b) 4 equ. Fmoc-AA-OH/HATU/HOAT, 8 equ. DIPEA, 30 minutes RT, c) 94% TFA, 2.5% water, 2.5% TIPS, 1% DODT, 3 hours. FIG. 2C: HPLC chromatograms and MS spectra of the crude peptide 3 (below) and the crude peptide after reaction with 2-(bromomethyl)acrylate (top). The second peak is excess 2-(bromomethyl)acrylate.

FIGS. 3A-3B. Designed methacrylate peptides bind 14-3-3σ. FIG. 3A. Peptides (200 μM) were incubated with the 14-3-3σ protein (2 μM; overnight; 4° C.) and analyzed using intact protein LC/MS. See Table 1 for peptide structures. FIG. 3B. Selected peptides (5 μM) were incubated with 14-3-3σ protein (2 μM, room temperature) for different times and analyzed using intact protein LC/MS. 3 and 8 formed only full peptide adducts. 11 also formed a transient methacrylate only adduct which converted to the full adduct over time.

FIGS. 4A-4B. Methacrylate peptides bind conserved 14-3-3 lysine residue. FIG. 4A: Overlay of the CovPepDock prediction and co-crystal structure of peptide SEQ ID 8 bound to 14-3-3σ (white surface; left), and a close-up view on the methacrylate residue (right). Final 2Fo-Fc electron density (light mesh, contoured at 1.0σ) is displayed for peptide SEQ ID 8 (right). Similarly, FIG. 4B: docking overlay (left) and close-up view (right) of peptide SEQ ID 3. Final 2Fo-Fc electron density (light mesh, contoured at 1.0σ) is displayed for peptide (right)

FIGS. 5A-5B. Labeling of 14-3-3 proteins by BODIPY-modified peptides in A549 lysates and in medium. A549 cells were grown for the indicated times in serum-free media. Lysates (FIG. 5A) or concentrated media (FIG. 5B) were then incubated for various times at room temperature with the peptides, followed by SDS-PAGE and western blot. Bottom Panel: Detection of 14-3-3β by western blot; Middle panel—peptide fluorescence; right panel—overlaid images. The Y-axis refers to molecular weight.

FIG. 6. Characterization of the selectivity of peptides SEQ ID 3 and SEQ ID 8 using pull-down proteomics. A549 lysates were treated with biotinylated derivatives of peptides SEQ ID 3 or SEQ ID 8 (1 μM, 22 hours 25° C.). The biotinylated proteins were enriched using streptavidin beads, digested with trypsin and analyzed using LC-MS/MS.

FIGS. 7A-7D. Generation of an Im9 protein that can irreversibly bind E9. FIG. 7A: Model of the C23A/E41C mutant of Im9 covalently bound to Lys97 in E9 (white), compared to the wild type complex (with Im9). FIG. 7B: Deconvoluted MS spectra of purified Im methacrylate, purified E9 and the covalent complex formed after their incubation. FIG. 7C: Reverse phase HPLC chromatograms of samples of Im methacrylate incubated with wild type E9 and several E9 mutants. Incubation with the K97R mutant abolishes covalent bond formation. FIG. 7D: Deconvoluted mass spectra of Im C23A/E41C mutant (Top) and the methacrylate-modified protein (bottom).

FIGS. 8A-8D. Methacrylate peptides label different 14-3-3σ residues. FIG. 8A: Peptide SEQ ID 3 was incubated with 2 mM of either TCEP or DTT for 130 minutes at room temperature and the products were characterized using LC/MS. FIG. 8B: 14-3-3σ was incubated with peptide SEQ ID 3 until fully labeled, followed by incubation with TCEP or DTT for 130 minutes at room temperature and analysis by LC/MS. FIG. 8C: 14-3-3σ was incubated with Bodipy-modified peptides (SEQ ID 3 and SEQ ID 8) until fully labeled. The samples were then denatured using Lithium Dodecyl Sulfate buffer in different conditions (with or without heating and with or without DTT) and analyzed by SDS-PAGE. (See Example 2). FIG. 8D: Following incubation with the peptides (SEQ ID 3, SEQ ID 8 and SEQ ID 11), samples of 14-3-3σ were trypsinized and tryptic peptides (numbered 1-9-SEQ ID 29-37, respectively) containing or following potential target residues were quantified using parallel reaction monitoring.

FIG. 9. Fluorescence Polarization competition experiments. Fluorescence polarization of 5 nM BODIPY-labeled noncovalent binder for 14-3-3σ was measured alone (SEQ ID 38), in the presence of 0.25 μM 14-3-3σ and in the presence of 14-3-3σ and the different electrophilic peptides (SEQ ID 1-11 5 μM and 200 μM). The sequence of the BDP labeled binder is shown on top (SEQ ID 38), Dab=diaminobutyric acid. The control peptide is identical to the BDP-labeled binder in sequence.

FIG. 10. Peptide labeling at various temperatures. Peptides SEQ ID 3 and SEQ ID 8 (5 μM) were incubated with 14-3-3σ (2 μM) at either 25° C. or 37° C. and the degree of labeling was measured using LC/MS.

FIGS. 11A-11B. Covalent complex formation significantly stabilizes complex thermal stability. FIG. 11A: Average derivative data from 3 samples is shown for each condition. FIG. 11B: measured melting temperature (calculated from maximum derivative) for different constructs. Error bars represent standard deviation (n=3). Melting temperatures could not be calculated for E9 constructs alone which displayed no clear melting curve.

FIGS. 12A-12B. Alternative electrophiles can tune the reaction kinetics. FIG. 12A: Labeling kinetics of 14-3-3σ (2 μM) by peptide SEQ ID 3 (ethyl ester-Peptide 3 and phenyl ester-Peptide 3a) at 25° C., 5 μM peptide. FIG. 12B: CovPepDock model of peptide 3a bound to 14-3-3σ overlaid on the structure of parent noncovalent peptide-.

FIGS. 13A-13B. Structural insights provided by CovPepDock for covalent protein reagents. FIG. 13A. Top-scoring model of the E9-Im9 complex when mutating Val34 of Im9 to the methacrylate warhead. While this position is located in close proximity to the target Lys97 (Cα-Cα distance=7.5 Å), none of the 10 top-scoring models had constraint score<2, indicating a non-ideal covalent bond geometry. FIG. 13B. Docking model (RMSD=0.758 Å) of the E9-Im9 complex when mutating Glu42. While this position seems to be pointing away from the target Lys97, CovPepDock revealed a potential shift of the helix that enables the formation of the covalent bond.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Modified Protein or Modified Peptide

In some embodiments, provided herein a modified protein or a modified peptide comprising a synthetic/recombinant protein or a synthetic peptide modified by an electrophile, wherein the electrophile is covalently bound to a thiol group of a cysteine within the synthetic/recombinant protein or synthetic peptide and the electrophile is represented by the structure of Formula I.

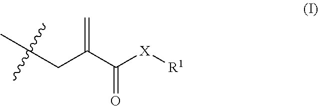

wherein,
R¹ comprises substituted or unsubstituted alkyl, alkenyl, alkynyl, carbocycle, aryl, heteroaryl or heterocyclic; and
X comprises O, NR³ or substituted or unsubstituted alkylene; and R³ is hydrogen, substituted or unsubstituted alkyl, alkenyl, alkynyl, carbocycle, aryl, heteroaryl or heterocyclic; wherein the modified protein or modified peptide is capable of covalently binding a target protein.

In other embodiments, the electrophile is represented by the structure of formula II (methacrylate):

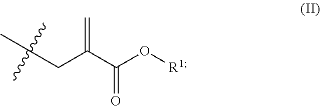

wherein, R¹ comprises substituted or unsubstituted alkyl, alkenyl, alkynyl, carbocycle, carbocycle, aryl, heteroaryl or heterocyclic.

In other embodiments, the electrophile is:

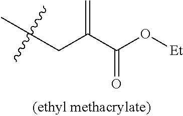 [SMILES: *CC(=C)C(=O)OCC]

In other embodiments, the electrophile is represented by the structure of formula III (methacrylamide):

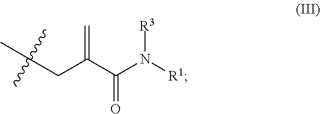

wherein, each of R¹ or R³ comprises substituted or unsubstituted alkyl, alkenyl, alkynyl, aryl, carbocycle, heteroaryl or heterocyclic.

In other embodiments, the electrophile is represented by the structure of formula IV:

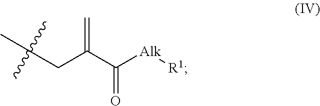

wherein, R¹ comprises substituted or unsubstituted alkyl, alkenyl, alkynyl, aryl, carbocycle, heteroaryl or heterocyclic; and Alk refers to substituted or unsubstituted alkylene.

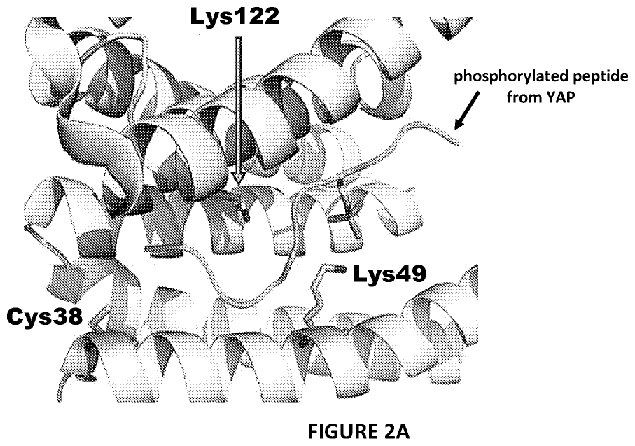

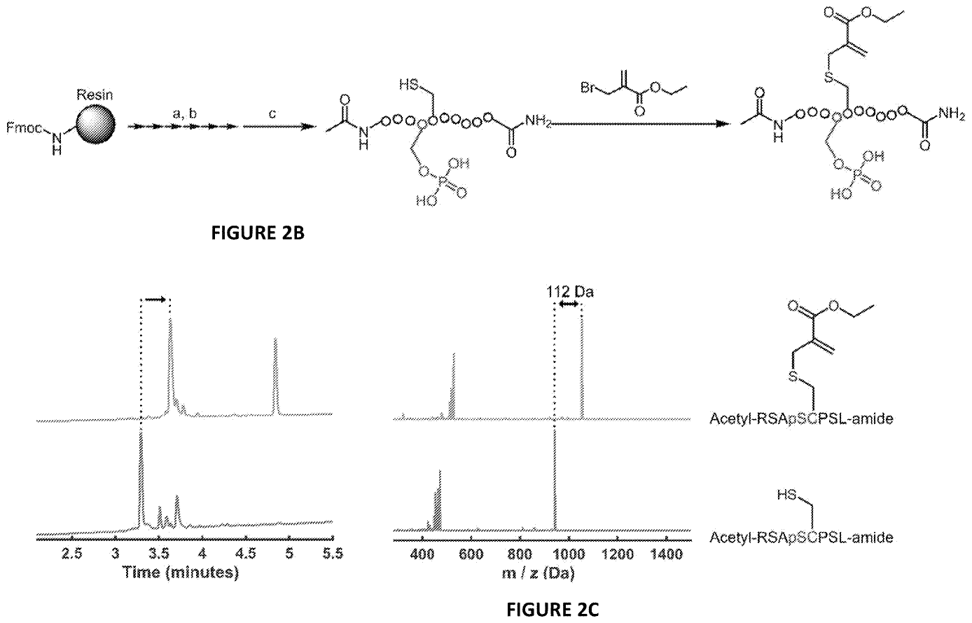

In some embodiments, the modified protein or modified peptide comprise: (i) an electrophile installation on unprotected peptides or proteins; (ii) the electrophile reacts with both thiols and primary amines of a target protein. As such the electrophile of formula I-IV is selected that react with both cysteine, and lysine residues of the target protein via aza-Michael addition. As a nonlimiting example ethyl 2-(bromomethyl)acrylate (FIG. 2B) is used for its selective reactivity with cysteine residues via its α-bromo-methylene functional group.

In some embodiments, provided herein a modified protein or a modified peptide comprising a synthetic/recombinant protein or a synthetic peptide modified by an electrophile of Formula I-IV, wherein the electrophile is covalently bound to a thiol group of a cysteine within the synthetic/recombinant protein or synthetic peptide. The cysteine residues represent an attractive anchoring residue due to their low proteomic abundance and their high reactivity at physiological pH, enabling rapid and selective modification on unprotected peptides and proteins.

In some embodiments, provided herein a modified protein or a modified peptide comprising a synthetic/recombinant protein or a synthetic peptide modified by an electrophile of Formula I-IV, forming a thioether bond between the electrophile and the synthetic/recombinant protein or synthetic peptide.

In some embodiments, provided herein a modified protein comprising a synthetic protein modified by an electrophile of Formula I-IV, forming a thioether bond between the electrophile and the synthetic protein.

In some embodiments, provided herein a modified protein comprising a recombinant protein modified by an electrophile of Formula I-IV, forming a thioether bond between the electrophile and the recombinant protein.

In some embodiments, provided herein a modified peptide comprising synthetic peptide modified by an electrophile of Formula I-IV, forming a thioether bond between the electrophile and the synthetic peptide.

In some embodiments, the modified protein or modified peptide is capable of covalently binding a target protein, having a noncovalent recognition with the target protein prior to the covalent binding with the target protein.

In some embodiments, the modified protein or modified peptide is capable of covalently binding a target protein, wherein the electrophile is in close proximity to a target residue of the target protein prior to the covalent binding with the target protein.

In some embodiments, the modified protein or modified peptide is capable of covalently binding a target protein, wherein the modified protein or modified peptide has a non-covalent recognition with the target protein; and the electrophile is in close proximity to a target residue of the target protein; prior to the covalent binding with the target protein. In other embodiments, the target residue within the target protein is a cysteine residue (SH) or a lysine residue (NH₂). In other embodiments, the close proximity between the electrophile and the target residue within the target protein refers to a distance being less than 15 Å. In other embodiments less than 14 Å, 13 Å, 12 Å, 11 Å, 10 Å, 7 Å, or 5 Å. In other embodiments a distance of between 5 to 15 Å, 2 to 7 Å, 2 to 7 Å, 5 to 10 Å.

In some embodiments, the modified protein or modified peptide described herein covalently binds a target protein via the double bond (C═CH₂) of the electrophile of Formula I-IV to a thiol or an amine within the target protein. In other embodiments, the modified protein or modified peptide described herein covalently binds the thiol (SH) side chain of a cysteine within the target protein. In other embodiments, the modified protein or modified peptide described herein covalently binds the amine side chain (NH₂) of a lysine within the target protein. In other embodiments, the modified protein or modified peptide covalently binds the double bond (C═CH₂) of the electrophile of Formula I-IV and a lysine residue within the target protein via aza-Michael addition. In other embodiments, the modified protein or modified peptide covalently binds the double bond (C═CH₂) of the electrophile of Formula I-IV and a cysteine residue within the target protein.

The electrophilic peptides or proteins (i.e the modified proteins or modified peptides) described herein provide a versatile approach to convert native peptide sequences or native proteins into covalent binders that can target a broad range of residues, and can be installed easily on unprotected peptides and proteins via cysteine side chains, and react efficiently and selectively with cysteine and lysine side chain on the target protein.

In some embodiments, the modified peptide provided herein is Ac-RSApSmCPSL-NH₂ (SEQ ID 3), wherein pS is Phoshoserine and mC is Methacrylate-modified cysteine. In some embodiments, the modified peptide provided herein is Ac-RAHpSmCPASLQ-NH₂ (SEQ ID 8), wherein pS is Phoshoserine and mC is Methacrylate-modified cysteine. In some embodiments, the modified peptide provided herein is Ac-RAHpSSPASmCQ-NH₂ (SEQ ID 11), wherein pS is Phoshoserine and mC is Methacrylate-modified cysteine.

In other embodiments, the modified peptide Ac-RSApSmCPSL-NH₂ (SEQ ID 3), Ac-RAHpSmCPASLQ-NH₂ (SEQ ID 8) or Ac-RAHpSSPASmCQ-NH₂ (SEQ ID 11), covalently binds 14-3-3 as a target protein. In other embodiments, the modified peptide Ac-RSApSmCPSL-NH₂(SEQ ID 3), Ac-RAHpSmCPASLQ-NH₂ (SEQ ID 8) or Ac-RAHpSSPASmCQ-NH₂ (SEQ ID 11), covalently binds a Cys 38, Lys 122 or Lys 49 of the 14-3-3σ target protein, depending on the position of the electrophile.

The modified peptide Ac-RSApSmCPSL-NH₂ (SEQ ID 3), Ac-RAHpSmCPASLQ-NH₂ (SEQ ID 8) or Ac-RAHpSSPASmCQ-NH₂ (SEQ ID 11), covalently bound 14-3-3 as a target protein targeting a conserved lysine residue exhibiting broad reactivity against 14-3-3 proteins, and efficiently labeled 14-3-3 proteins in lysates, as well as secreted 14-3-3 extracellularly. The irreversible binding to the predicted target lysines were confirmed by proteomics and X-ray crystallography of the complexes.

In some embodiments, the modified protein provided herein is a modified immunity protein MELKHSISDYTEAEFLQLVTTIANADTSSEEELVKLVTHF(mC)EMTEHPSGSDLIYYPKEG DDDSPSGIVNTVKQWRAANGKSGFKQGLEHHHHHH (Im9, SEQ ID 27), wherein mC is Methacrylate-modified cysteine. The Alanine at position 23 was a cysteine in the original sequence of Im. The mC was a glutamate in the original sequence of Im. In other embodiments, the modified immunity protein (Im9, SEQ ID 27), covalently binds the E9 target protein.

The immunity protein irreversibly bound to the E9 DNAse, resulting in significantly higher thermal stability relative to the non-covalent complex.

The approach provided herein offers a simple and versatile route to convert peptides and proteins into potent covalent binders.

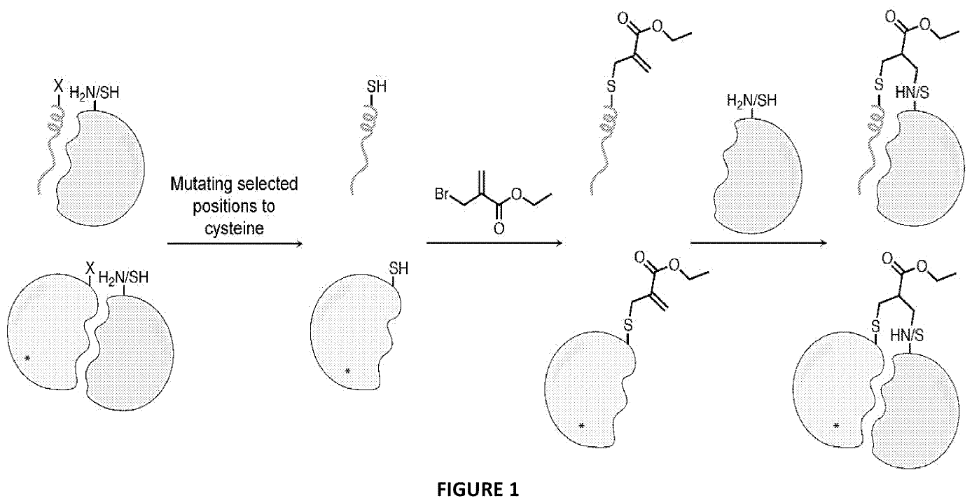

Provided herein a new approach to the development of covalent protein binders based on thioether-modified proteins or modified peptides. The modified protein or modified peptide can react with both lysine and cysteine side chains via Michael addition of a target protein. The electrophile of Formula I-IV is installed by direct, selective modification of a cysteine side chain, enabling synthesis of binders of unprotected peptides and even recombinant proteins (FIG. 1).

### Preparation of the Modified Protein or Modified Peptide

In some embodiments, provided herein a method of preparing a modified protein or a modified peptide described herein, wherein the method comprises;
a. identifying a target protein;
b. designing a peptide or protein covalent binder candidate based on previously characterized noncovalent binders of the target protein;
c. synthesizing a modified peptide or a modified protein, to allow recognition with the target protein at the recognition site; wherein said modified protein or modified peptide comprise a cysteine which is reacted with an electrophile of Formula I-IV.

In some embodiments, the design of the peptide or the protein covalent binder candidate is done by computational modeling, structure based rational design, random screening of possible positioning for modification, or any combination thereof. In other embodiments, the designed covalent binder candidate has molecular recognition with a target protein. The “peptide or protein covalent binder candidate” is prepared and modified with an electrophile of Formula I-IV to obtain the modified protein or modified peptide described herein.

The design of the peptide or protein covalent binder candidate is based on previously characterized noncovalent binders with a target protein. “Previously characterized noncovalent binders of the target protein refer to peptide-protein or protein-protein interactions known in the art, for example: PDB entries for known interactions. The term “noncovalent recognition” with a target protein refers to van-der walls interactions, π-π or hydrogen bonding between the modified protein or modified peptide with the target protein.

In some embodiments, based on the computer modeling, a synthetic protein is synthesized having only one cysteine amino acid which is further modified with the electrophile of Formula I-IV. In some embodiments, based on the modeling calculation, a synthetic peptide is synthesized having only one cysteine amino acid.

In some embodiments, the modified peptide is prepared according to FIG. 2B.

Computational modeling has been used extensively to model and design peptide and peptidomimetic binders for proteins. An example of a computational modeling system is the CovPepDock, Rosetta-based framework for modeling covalent protein-peptide interactions and for the design and virtual screening of potential covalent peptide binders (Tivon, B.; Gabizon, R.; Somsen, B. A.; Cossar, P. J.; Ottmann, C.; London, N. Covalent Flexible Peptide Docking in Rosetta. Chem. Sci. 2021, 12 (32), 10836-10847).

Using CovPepDock the modified peptides Ac-RSApSmCPSL-NH₂ (SEQ ID 3), Ac-RAHpSmCPASLQ-NH₂ (SEQ ID 8) or Ac-RAHpSSPASmCQ-NH₂ (SEQ ID 11), displayed highly potent and selective detection of 14-3-3 in cell lysates, a difficult task for noncovalent binders due to the high sequence homology within the family.

In some embodiments, the modified protein or modified peptide is prepared by reacting a synthetic/recombinant protein or synthetic peptide with an electrophilic reagent, wherein the synthetic/recombinant protein or synthetic peptide possess only one cysteine amino acid, and the electrophilic reagent reacts via the R² group (of the electrophilic reagent) with the thiol of the cysteine to form a thioether bond; wherein the electrophilic reagent is represented by the following structure if Formula IA, IIA, IIIA or IVA:

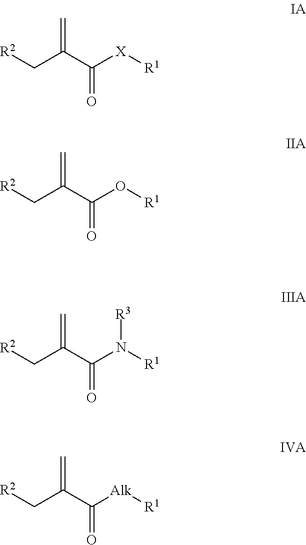

wherein,
R¹ comprises substituted or unsubstituted alkyl, alkenyl, alkynyl, carbocycle, aryl, heteroaryl or heterocyclic;
R² comprises Br, Cl or tosyl;
R³ comprises substituted or unsubstituted alkyl, alkenyl, alkynyl, carbocycle, aryl, heteroaryl or heterocyclic;
and
X comprises O, NR³ or substituted or unsubstituted alkylene.

In some embodiments, the electrophilic reagent is ethyl 2-(bromomethyl)acrylate.

In some embodiments, R¹ of Formula I, II, III, IV, IA, IIA, IIIA or IVA is substituted or unsubstituted alkyl, alkenyl, alkynyl, carbocycle, aryl, heteroaryl or heterocyclic. In other embodiments R¹ is substituted or unsubstituted alkyl. In other embodiments R¹ is substituted or unsubstituted alkenyl. In other embodiments R¹ is substituted or unsubstituted alkynyl. In other embodiments R¹ is substituted or unsubstituted carbocycle. In other embodiments R¹ is substituted or unsubstituted aryl. In other embodiments R¹ is substituted or unsubstituted phenyl. In other embodiments, R¹ is phenyl and the electrophile is a phenyl of formula II or IIA. In other embodiments R¹ is substituted or unsubstituted heteroaryl. In other embodiments R¹ is substituted or unsubstituted heterocyclic.

In some embodiments, R² of Formula IA, HA, IIIA or IVA is Br, Cl or tosyl. In other embodiments, R² is Cl. In other embodiments, R² is Br. In other embodiments, R² is tosyl.

In some embodiments, X of Formula I or IA is O, NR³ or substituted or unsubstituted alkylene. In other embodiments X is O. In other embodiments X is NR³. In other embodiments X is NH. In other embodiments X is substituted or unsubstituted alkylene. In other embodiments X is methylene (—CH₂—).

In some embodiments, R³ of Formula I, IA, III or IIIA is substituted or unsubstituted alkyl, alkenyl, alkynyl, carbocycle, aryl, heteroaryl or heterocyclic. In other embodiments R³ is substituted or unsubstituted alkyl. In other embodiments R³ is substituted or unsubstituted alkenyl. In other embodiments R³ is substituted or unsubstituted alkynyl. In other embodiments R³ is substituted or unsubstituted carbocycle. In other embodiments R¹ is substituted or unsubstituted aryl. In other embodiments R³ is substituted or unsubstituted heteroaryl. In other embodiments R³ is substituted or unsubstituted heterocyclic.

As used herein, “alkyl” refers to a linear or branched alkyl. In certain embodiments linear or branched alkyl having from 1 to about 20 carbon atoms, in another embodiment having from 1 to 12 carbons. In a further embodiment the alkyl includes lower alkyl, having 1 to 6 carbons. In a further embodiment the alkyl includes lower alkyl, having 1 to 3 carbons. There may be optionally inserted along the alkyl group one or more oxygen, sulfur, including S(═O) and S(═O)₂ groups, or substituted or unsubstituted nitrogen atoms including —NR— and —N⁺RR— groups, where the nitrogen substituent(s) is(are) alkyl, aryl, aralkyl, heteroaryl, heteroaralkyl or COR, wherein each R is independently selected from alkyl, aryl, aralkyl, heteroaryl, heteroaralkyl, —OY or —NYY, wherein each Y is independently selected from hydrogen, alkyl, aryl, heteroaryl, cycloalkyl or heterocyclyl. The alkyl groups include, but are not limited to, methyl, ethyl, propyl, methoxy, ethoxy, isopropyl, isobutyl. The alkyl group may be substituted or unsubstituted. In other embodiments the substituted alkyl may be substituted with one or more (e.g., one, two, three, or more, as valency allows) groups independently selected from halo, hydroxy, amino, COOH, alkoxy, cyano, oxo, aryl, nitro, azide, aryloxy, sulfinyl, sulfonyl, sulfonate, sulfate, carbonyl, amine or amide.

As used herein, “alkylene” refers to a linear, branched or cyclic, in certain embodiments linear or branched, divalent aliphatic hydrocarbon group, in one embodiment having from 1 to about 20 carbon atoms, in another embodiment having from 1 to 12 carbons. In a further embodiment alkylene includes lower alkylene having 1 to 6 carbons. In a further embodiment alkylene includes lower alkylene having 1 to 3 carbons. There may be optionally inserted along the alkylene group one or more oxygen, sulfur, including S(═O) and S(═O)₂ groups, or substituted or unsubstituted nitrogen atoms including —NR— and —N⁺RR— groups, where the nitrogen substituent(s) is(are) alkyl, aryl, aralkyl, heteroaryl, heteroaralkyl or COR, wherein each R is independently selected from alkyl, aryl, aralkyl, heteroaryl, heteroaralkyl, —OY or —NYY, wherein each Y is independently selected from hydrogen, alkyl, aryl, heteroaryl, cycloalkyl or heterocyclyl. Alkylene groups include, but are not limited to, methylene (—CH₂), ethylene (—CH₂CH₂—), propylene (—(CH₂)₃), methylenedioxy (—O—CH₂—O—) and ethylenedioxy (—O—(CH₂)₂—O—).

As used herein, “alkenyl” refers to a linear or branched alkenyl. In one embodiment straight or branched alkynyl having from 2 to about 20 carbon atoms and at least one double bond, in other embodiments 1 to 12 carbons. In further embodiments, alkenyl groups include lower alkenyl having 2 to 6 carbons. In further embodiments, alkenyl groups include lower alkenyl having 3 to 4 carbons. There may be optionally inserted along the alkenyl group one or more oxygen, sulfur or substituted or unsubstituted nitrogen atoms, where the nitrogen substituent is alkyl. Alkenyl groups include, but are not limited to, —CH═CH—CH═CH₂ and —H═CH—CH₃. The alkenyl group may be substituted or unsubstituted. In other embodiments the substituted alkenyl may be substituted with one or more (e.g., one, two, three, or more, as valency allows) groups independently selected from halo, hydroxy, amino, COOH, alkoxy, cyano, oxo, aryl, nitro, azide, aryloxy, sulfinyl, sulfonyl, sulfonate, sulfate, carbonyl, amine or amide.

As used herein, “alkynyl” refers to a straight or branched alkynyl. In certain embodiments straight or branched alkynyl, in one embodiment having from 2 to about 20 carbon atoms and at least one triple bond, in another embodiment 1 to 12 carbons. In a further embodiment, alkynyl includes lower alkynyl having 2 to 6 carbons. In a further embodiment, alkynyl includes lower alkynyl having 3 to 4 carbons. There may be optionally inserted along the alkynyl group one or more oxygen, sulfur or substituted or unsubstituted nitrogen atoms, where the nitrogen substituent is alkyl. Alkynyl groups include, but are not limited to, —C≡C—C≡CH, —C≡CH and —C≡C—CH₃. The alkynyl group may be substituted or unsubstituted. In other embodiments the substituted alkynyl may be substituted with one or more (e.g., one, two, three, or more, as valency allows) groups independently selected from halo, hydroxy, amino, COOH, alkoxy, cyano, oxo, aryl, nitro, azide, aryloxy, sulfinyl, sulfonyl, sulfonate, sulfate, carbonyl, amine or amide.

A “carbocycle” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of carbocycle groups are cyclopropane, cyclobutane, cyclopentane, cyclopentene, cyclohexane, cycloheptane, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, amino, COOH, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, hydrazine, hydrazide, thiohydrazide, and amino. When a carbocycle group is unsaturated, it may comprise at least one carbon-carbon double bond and/or at least one carbon-carbon triple bond. An unsaturated carbocycle include cyclohexene, cycloheptene, cyclohexadiene, cycloheptatriene.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, amino, COOH, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, 0-carbamyl, N-carbamyl, O-thiocarbamyl, N-thiocarbamyl, C-amido, N-amido, C-carboxy, O-carboxy, sulfonamido, hydrazine, hydrazide, thiohydrazide, and amino.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, amino, COOH, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, 0-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

A “heterocyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heterocyclic may be substituted or non-substituted. When substituted, the substituted group can be, for example, alkyl, alkenyl, alkynyl, cycloalkyl, aryl, heteroaryl, heteroalicyclic, halo, hydroxy, amino, COOH, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, hydrazine, hydrazide, thiohydrazide, and amino. Representative examples are piperidine, piperazine, tetrahydrofuran, tetrahydropyran, morpholine and the like.

Uses of the Covalent Binding of the Modified Protein or Modified Peptide with a Target Protein

The covalent binding of the modified protein or modified peptide with a target protein provides high potency and specificity that can be useful as therapeutics and chemical biology tools.

In some embodiments, provided herein a protein-protein covalent conjugate comprising a modified protein provided herein, covalently bound to a target protein, wherein the modified protein and the target protein possess non-covalent recognition prior to the covalent binding with the target protein. In other embodiments, the protein-protein covalent conjugate comprises a modified protein of Im9 and a target protein E9. In other embodiments, the protein-protein covalent conjugate (of the Im9 and E9) displayed a higher thermal stability than the noncovalent complex.

In some embodiments, provided herein a peptide-protein covalent conjugate comprising a modified peptide provided herein, covalently bound to a target protein, wherein the modified peptide and the target protein possess non-covalent recognition prior to the covalent binding with the target protein. In other embodiments, the modified peptide is Ac-RSApSmCPSL-NH₂ (SEQ ID 3), Ac-RAHpSmCPASLQ-NH₂ (SEQ ID 8) or Ac-RAHpSSPASmCQ-NH₂ (SEQ ID 11); wherein pS is Phoshoserine and mC is Methacrylate-modified cysteine; and the target protein is 14-3-3.

In some embodiments, the target protein is 14-3-3.

In some embodiments, the target protein is 14-3-3 sigma represented by the following sequence.

(SEQ ID 28)
SYYHHHHHHDYDIPTTENLYFQGAMGSMERASLIQKAKLAEQAERY
EDMAAFMKGAVEKGEELSCEERNLLSVAYKNVVGGQRAAWRVLSS
IEQKSNEEGSEEKGPEVREYREKVETELQGVCDTVLGLLDSHLIK
EAGDAESRVFYLKMKGDYYRYLAEVATGDDKKRIIDSARSAYQEA
MDISKKEMPPTNPIRLGLALNFSVFHYEIANSPEEAISLAKTTFD
EAMADLHTLSEDSYKDSTLIMQLLRDNLTLWTADNAGEEGGEAPQ
EPQS.

Peptides derivatized with covalent warheads can greatly expand the repertoire of addressable targets. Provided herein an electrophilic reagent of Formula IA, IIA, IIIA or IVA which binds a peptide or protein to yield a modified peptide/protein (an electrophile peptide/protein). Such electrophilic peptide/protein: bind target proteins, covalently and selectively and even repurposed to enable modifications such as fluorescent labeling, drug conjugation or conjugation to other proteins. Provided herein an approach enabling the modification of native peptides or proteins by an electrophile of Formula IA, IIA, IIIA or IVA. This approach is differentiated in two respects. First, is the facile synthesis that does not require non-canonical amino-acids and incorporates the electrophile in a single step on a native cysteine residue (FIGS. 2A-2C). Second, we do not require non-canonical amino-acids and can use ‘generic’ SPPS (solid-phase peptide synthesis), the peptides are synthesized with a ‘canonical’ cysteine at the electrophilic site, therefore, this approach is used for the synthesis of recombinant proteins. Furthermore, the modified protein and modified peptide of this invention react in addition to cysteines, also with lysines of the target protein.

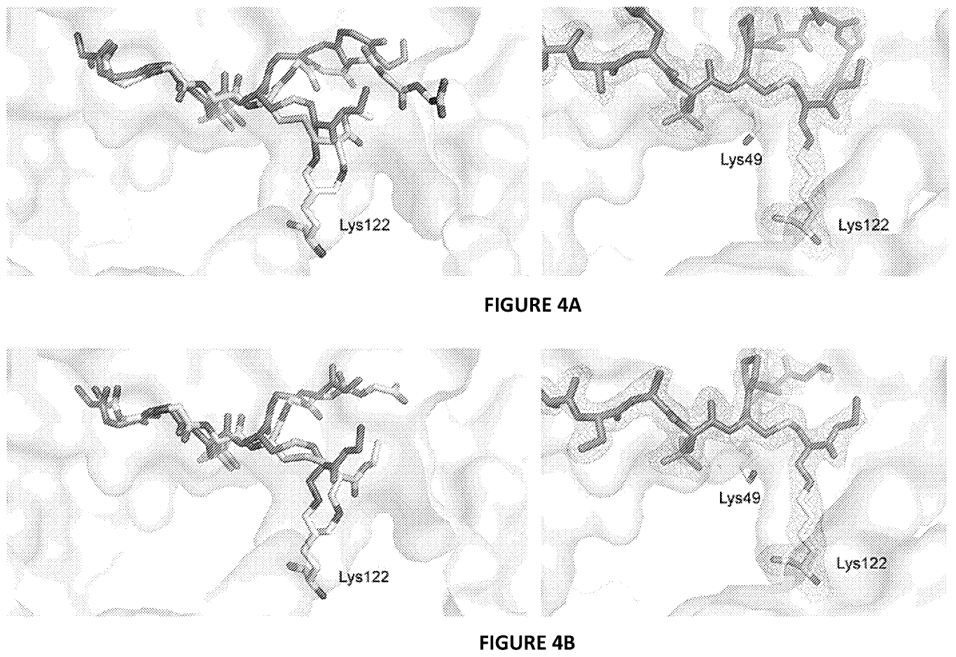

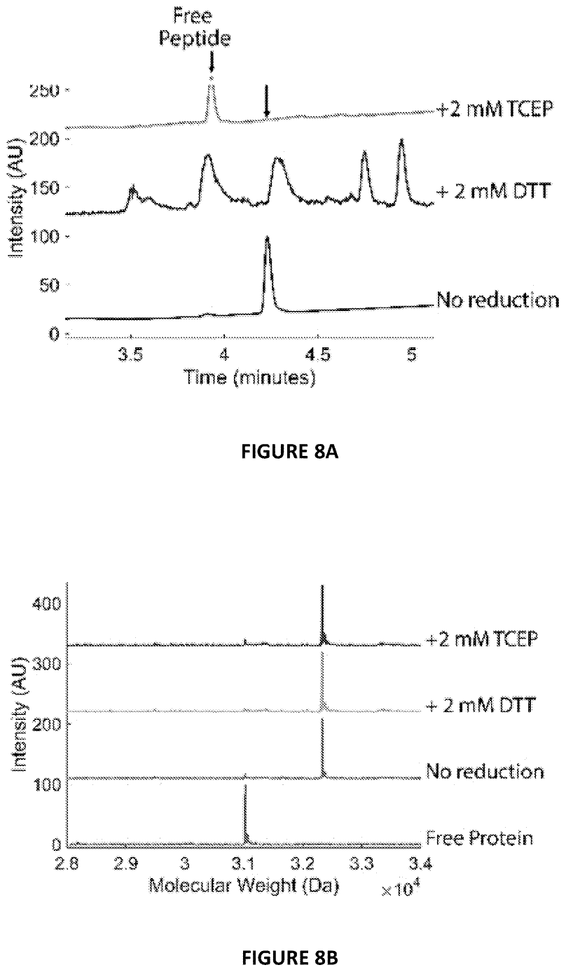

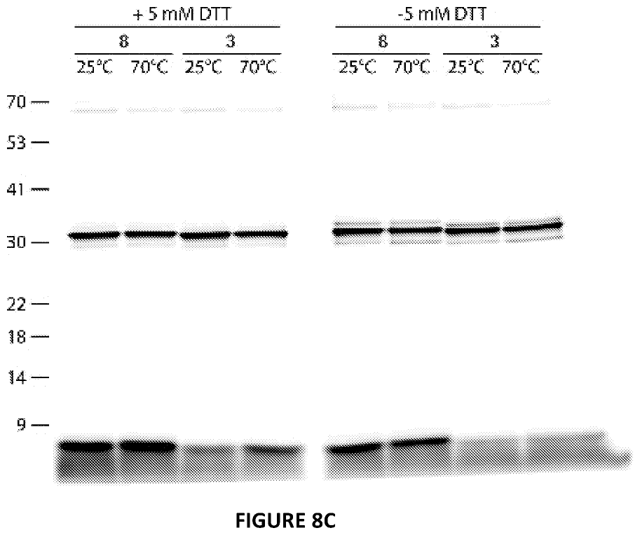

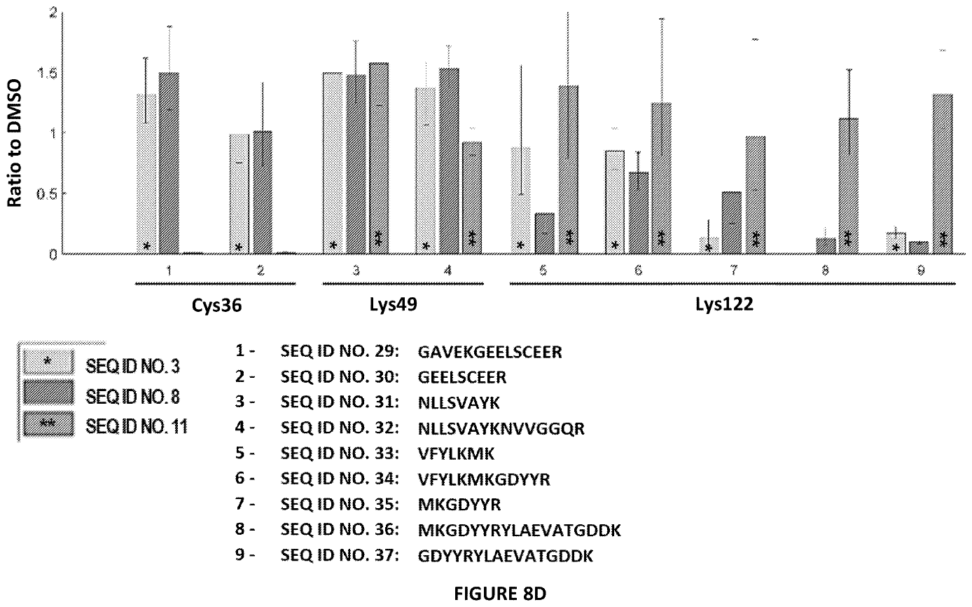

An advantage of this property is that such chemistry can be incorporated into covalent phage-display platforms for efficient discovery of electrophilic peptides Second, very few peptides were previously designed to irreversibly target lysine residues and the few that did, utilized aryl sulfonyl fluorides. Provided herein the first electrophilic peptides employing methacrylates to irreversibly label lysine target residues, as validated by crystallography (FIGS. 4A-4B) and mass spectrometry (FIGS. 8A-8D).

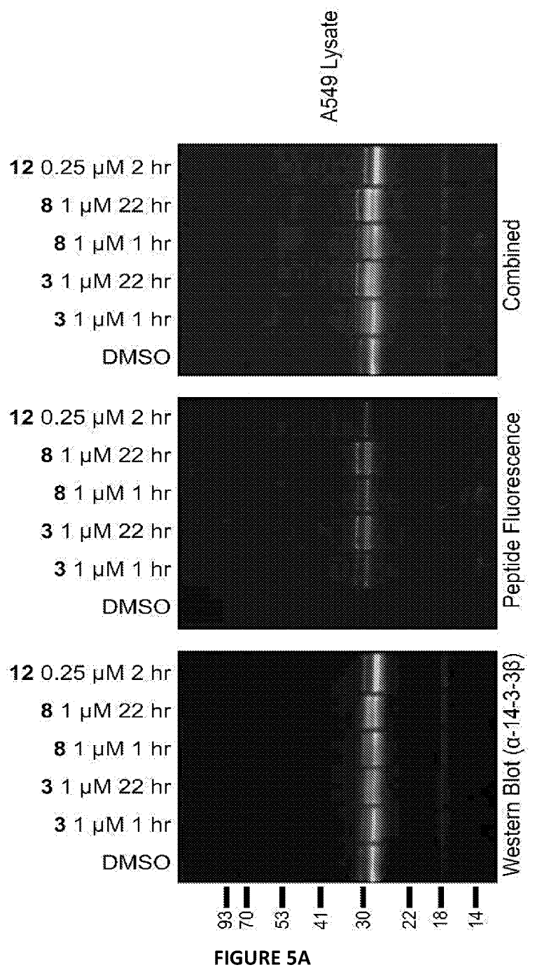

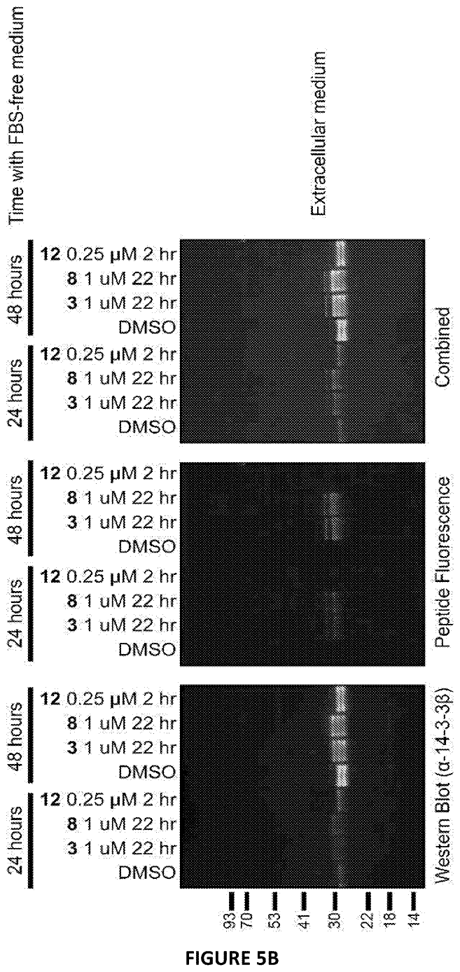

The potency and specificity of the peptides make them versatile chemical probes. While most known functions of 14-3-3 proteins are intracellular, they also serve some extracellular functions and the presence of extracellular 14-3-3 proteins can serve as a biomarker for various diseases. Therefore, the ability to bind and detect these proteins in extracellular media is of great interest. Using BDP-modified methacrylate peptides 14-3-3β was detected in extracellular medium with sensitivity equivalent or higher than western blot (FIGS. 5A-5B), illustrating the power of these probes. Furthermore, when targeting extracellular proteins, issues such as membrane permeability and proteolytic stability have less impact on the activity of the probe, opening the door for peptide and protein-based covalent probes.

Since the approach is applicable to peptides in their native form, it can be used to prepare electrophile-modified proteins directly from native, recombinant proteins. The resulting probes can efficiently and selectively label target proteins on cysteines and lysines. Furthermore, the R¹ group of the electrophile of Formula I-IV group can function as a point of diversification and screening either for the development of more potent binders or for the functionalization of the probes in various ways.

Genetic code expansion has previously enabled the installation of fluorosulfates on proteins to target a histidine residue. The approach provided herein offers a few advantages. First, genetic code expansion requires specialized bacterial expression systems and conditions that are not yet very widespread, somewhat limiting its broad applicability. Second, for future industrial applications such modified genetic systems might limit the scale of production, while canonical recombinant proteins with chemical modifications (such as Antibody-Drug-Conjugates) were already proven applicable. Third, for genetic code expansion, extensive work should be undertaken to enable the installation of a new type of amino acid. Thus, optimizing the features of the electrophile are limited. In the system described herein, many electrophilic reagents of Formula IA-IVA can be synthesized and conjugated to the same recombinant protein enabling much higher optimization throughput. Finally, the ethyl ester group can function not only as a point of diversification and screening for the development of better binders but also for various functionalization of the probes, for example via the attachment of E3 ligase binders for targeted degradation, for the attachment of fluorophores for imaging or detection purposes, for targeting proteins to particular cell types or locations in the cell and more.

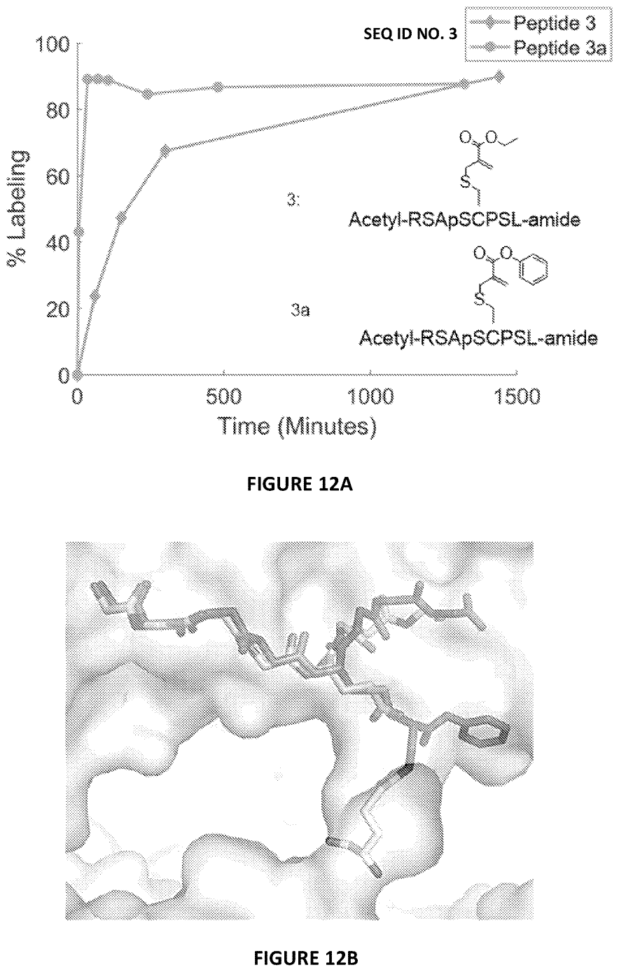

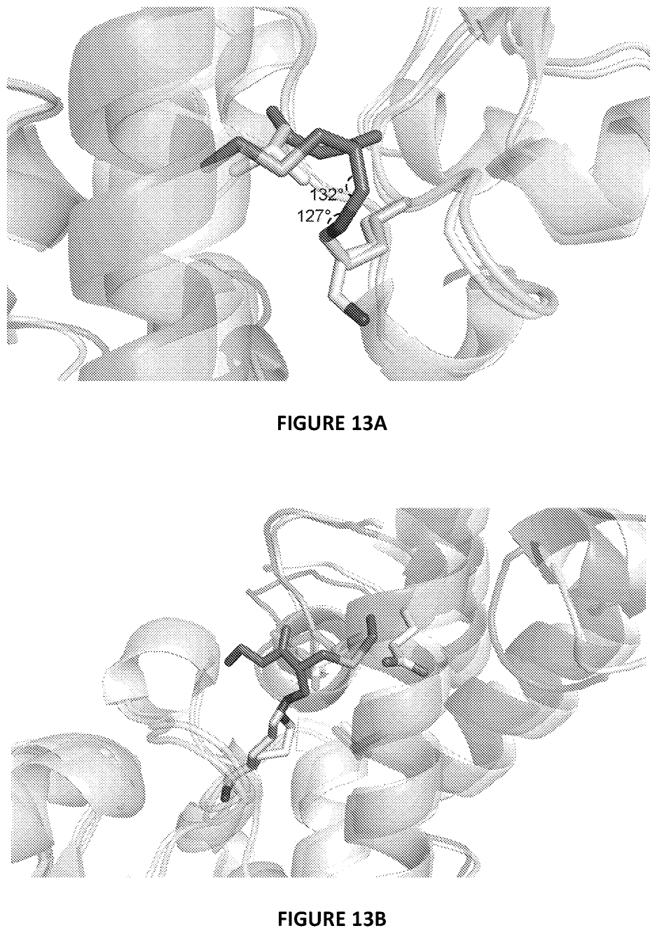

Another aspect that contributes to the practicality of the approach provided herein is the computational modeling support. While manual inspection and selection of positions for introduction of the electrophile, may work well for a few peptides, automatic modeling and selection can cover larger number of possibilities. Moreover, as the number of available electrophiles are expanded, with variable side-chains, modeling will be required to address the combinatorics (FIGS. 12A-12B). Finally, for protein covalent reagents (compared to peptides), manual inspection can be more challenging (FIGS. 13A-13B).

Provided herein a simple and versatile method for the preparation of a new class of covalent protein and peptide probes or covalent protein and protein probes suitable to bind a large variety of biological targets, and as such would support new applications in chemical biology and covalent drug discovery.

In some embodiments, provided herein a modified protein or modified peptide for use in selectively label, fluorescent label, inhibition, drug conjugation or conjugation to a target protein.

In some embodiments, provided herein a method of diagnosing a disease by a known biomarker wherein the method comprises covalently binding a modified peptide or modified protein described herein with a known biomarker, thereby identifying the biomarker by fluorescence or immunoassays.

The potency and specificity of the modified peptides or modified proteins can be used as versatile chemical probes. For example, while most known functions of 14-3-3 proteins are intracellular, they also serve some extracellular functions and the presence of extracellular 14-3-3 proteins can serve as a biomarker for various diseases. Using BDP (fluorescent dye Bodipy)—modified methacrylate peptides a 14-3-3 was detected in extracellular medium with higher sensitivity than western blot, illustrating the power of this method. (See Example 3). Furthermore, when targeting extracellular proteins, issues such as membrane permeability and proteolytic stability have less impact on the activity of the probe, opening the door for peptide and protein-based covalent probes.

The following examples are presented in order to more fully illustrate the preferred embodiments of the invention. They should in no way be construed, however, as limiting the broad scope of the invention.

### EXAMPLES

### Example 1

### Design and Synthesis of Modified Peptides Covalent Binders to 14-3-3 Proteins

The objective was to design binders that would target conserved lysines in the phoshopeptide binding groove of 14-3-3σ: lysines 49 and 122 (FIG. 2A). Specifically, Lys122 has been previously shown to react with aldehydes to form a reversible covalent imine bond, with high selectivity over other lysine residues, which is attributed to a lower pKa of the Lys122 side-chain (Wolter, M., et al., Fragment-Based Stabilizers of Protein-Protein Interactions through Imine-Based Tethering. Angew. Chem. Int. Ed Engl. 2020, 59 (48), 21520-21524.; Cossar, P. J. et al., Reversible Covalent Imine-Tethering for Selective Stabilization of 14-3-3 Hub Protein Interactions. J. Am. Chem. Soc. 2021, 143 (22), 8454-8464.; Wolter, M.; Valenti, D.; Cossar, P. J. et al., An Exploration of Chemical Properties Required for Cooperative Stabilization of the 14-3-3 Interaction with NF-κB-Utilizing a Reversible Covalent Tethering Approach. J. Med. Chem. 2021, 64 (12), 8423-8436.)

CovPepDock was first used to design a series of methacrylate-based peptides based on the non-covalent complex between 14-3-3σ and YAP1 phosphopeptide (Protein data bank (PDB): 3MHR).

The residues were identified in the YAP1 peptide that were within Cα-Cα distance<14 Å from the target lysine and mutated each of these residues to the methacrylate modified side-chain; this included residues 126-131 for targeting Lys49, and residues 126-133 for targeting Lys122. Four peptides were selected that were predicted to bind either Lys49 or Lys122 with high score and low Root Mean Square Deviation (RMSD) to the original peptide binding mode (i.e. predicted to bind well and maintain the binding pose of the original peptide). To further expand the scope of series, a second set of peptides were designed based on other known peptide binders of 14-3-3σ. Four such structures were selected, with peptides derived from Raf1 (Rapidly Accelerated Fibrosarcoma 1) (PDB: 3IQU and 4IEA), TASK-3 (TWIK-related acid-sensitive K+ channel 3) (PDB: 3P1N) and SNAI1 (PDB: 4QLI). For these peptides, Lys122 was focused on, which was more reactive towards electrophiles.

CovPepDock was used to model Lys122-targeting peptides based on each of these structures and selected seven additional peptides with high scores and low RMSD from this set for synthesis and testing. The structures of the peptides are detailed in Table 1.

TABLE 1
Sequences and structures of the peptides*

| Peptide | PDB | | |
|---|---|---|---|
| Source | (Protein | | |
| Protein | data bank) | Sequence | |
| SEQ ID 1 | SNAI1 | 4QLI_4 | Ac-SHpTmCPS-NH₂ |
| SEQ ID 2 | SNAI1 | 4QLI_5 | Ac-SHpTLmCS-NH₂ |
| SEQ ID 3 | Raf1 | 4IEA_5 | Ac-RSApSmCPSL-NH₂ |
| SEQ ID 4 | Raf1 | 4IEA_7 | Ac-RSApSEPmCL-NH₂ |
| SEQ ID 5 | Raf1 | 4IEA_8 | Ac-RSApSEPSmC-NH₂ |
| SEQ ID 6 | Raf1 | 3IQU_6 | Ac-QRSTpSmC-OH |
| SEQ ID 7 | TASK-3 | 3P1N_6 | Ac-KRRKpSmC-NH₂ |
| SEQ ID 8 | YAP-1 | 3MHR_5 | Ac-RAHpSmCPASLQ-NH₂ |
| SEQ ID 9 | YAP-1 | 3MHR_7 | Ac-RAHpSSPmCSLQ-NH₂ |
| SEQ ID 10 | YAP-1 | 3MHR_8 | Ac-RAHpSSPAmCLQ-NH₂ |
| SEQ ID 11 | YAP-1 | 3MHR_9 | Ac-RAHpSSPASmCQ-NH₂ |
| SEQ ID 12 | YAP-1 | 3MHR_Cl | Ac-RAHpSSPASLX-NH₂ |

*pS = Phoshoserine; pT = Phosphothreonine; mC = Methacrylate-modified cysteine (FIGS. 1, 2A-2C); X = γ-chloroacetamido-diaminobutyric acid.
In some embodiments, SEQ ID 1-12 may be acylated or non acylated. In some embodiments, SEQ ID 1-12 may be with an amine end group or an amide end group.

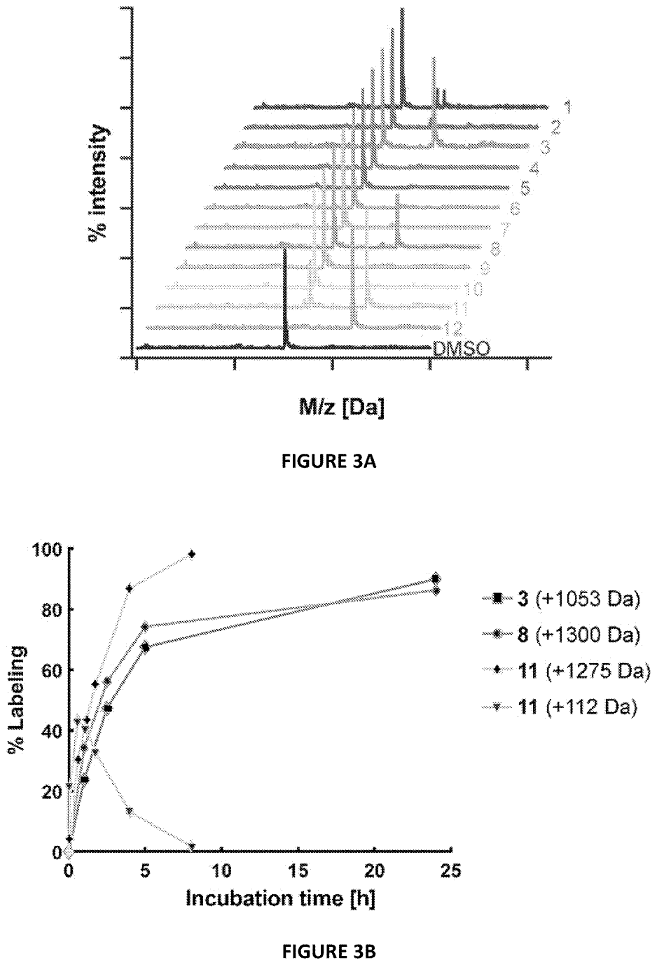

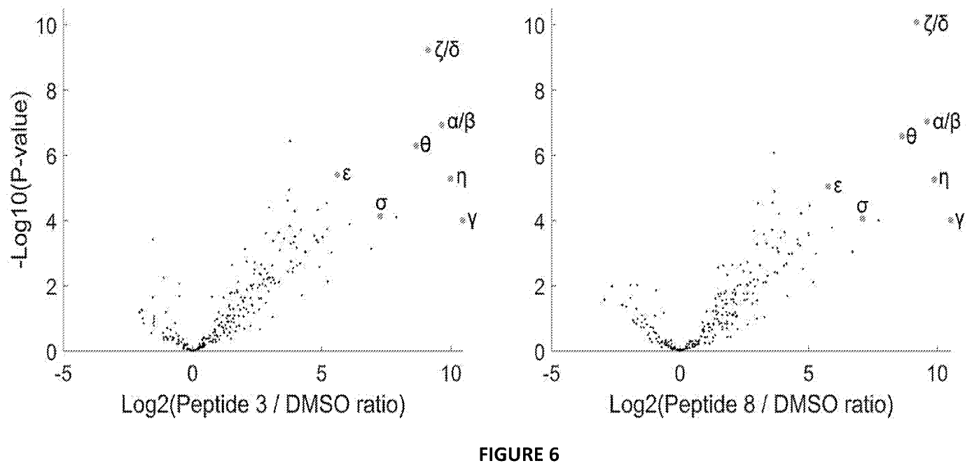

A covalent peptide docking pipeline CovPepDock was used to design candidate peptides based on known binding partners of 14-3-3σ (Table 1). This yielded 3 irreversible binding peptides out of 11 candidates. The hit peptide SEQ ID 11 reacted with Cys38 rather than the predicted Lys122. The electrophile in SEQ ID 11 is indeed closer to Cys38 in the binding pocket compared to the lysine targeting SEQ ID 8 (FIG. 2A). The reaction of SEQ ID 11 with the cysteine appears to occur via two distinct mechanisms—addition and substitution, which were also observed when the peptide is incubated with cysteine. In addition, the cysteine adds via Michael addition to the methacrylate, while in substitution, the cysteine displaces the peptide, which is released as a free thiol, while the methacrylate remains on the protein. The methacrylate-labeled protein can later react via addition with free thiol peptide, eventually converting all the protein to an addition product, which is stable (FIG. 3B). Reaction of the methacrylate peptides with thiols such as cysteine and DTT also releases free peptide (FIG. 8A). These results can be attributed to the higher nucleophilicity of the cysteine, which also reacts very rapidly with chloroacetamide-based peptides. Nevertheless, the methacrylate-based peptides are not promiscuous and display high specificity towards the target protein (FIGS. 5A-5B and 6) and target residue as indicated by single labeling even with excess peptide (100-fold excess; FIG. 3A). None of the peptides reacted with Lys49, which may be attributed to the lower predicted nucleophilicity of this residue.

To prepare peptide methacrylate adducts, the peptides were synthesized using standard Solid Phase Peptide Synthesis (SPPS) procedures and N-terminally acetylated the peptides (FIG. 2B). After cleavage from the resin, the crude peptides were reacted with 3 equivalents of 2-(bromomethyl)acrylate, in a buffer devoid of amines or thiols to avoid possible side reactions, resulting in efficient conversion of the peptide to the methacrylate (i.e. modified peptide) within 1-2 hours at room temperature (FIG. 2C).

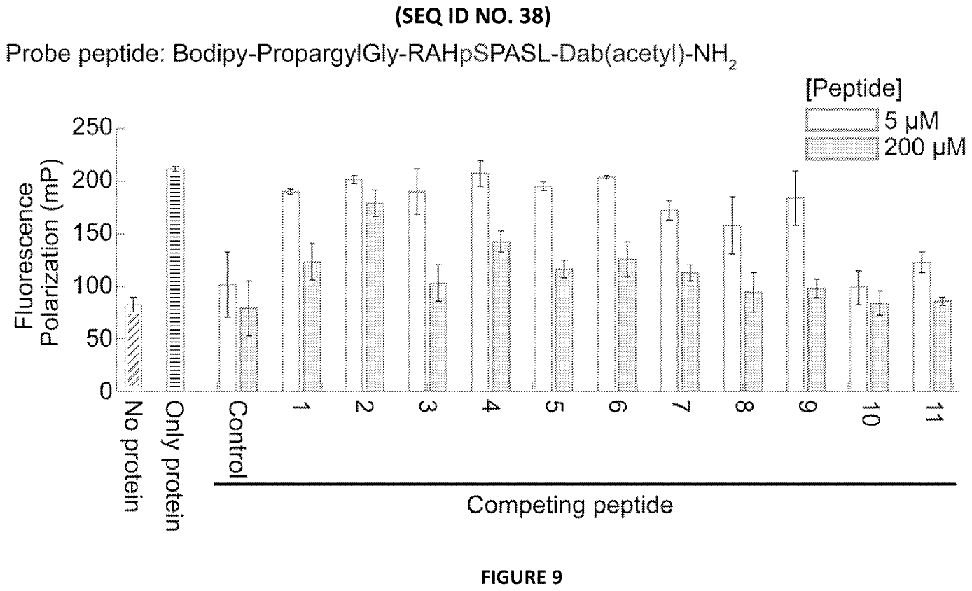

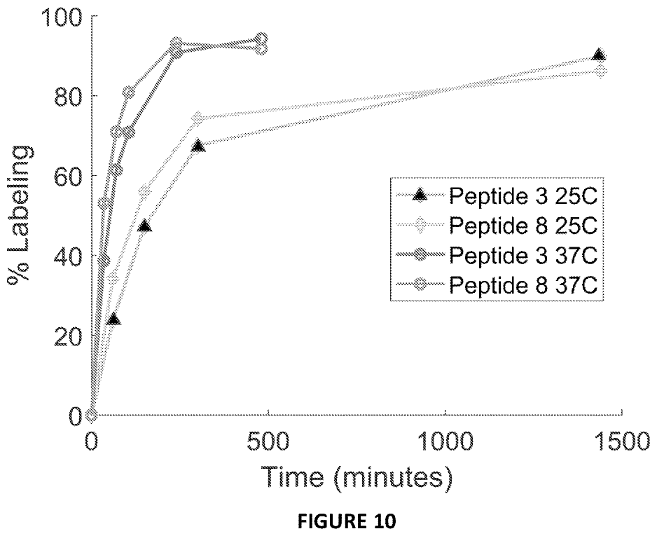

The peptides were incubated at 200 μM with 2 μM 14-3-3σ at 4° C. overnight and monitored the binding using intact protein LC/MS (FIG. 3A). Peptide SEQ ID 12, which contains a chloroacetamide warhead that reacts with 14-3-3σ via Cys38, was used as a positive control. Significant covalent labeling of 14-3-3σ with the expected adduct mass was observed for the peptides SEQ ID 3 (51%), SEQ ID 8 (35%) and SEQ ID 11 (11%), all of which were predicted to bind Lys122. Peptide SEQ ID 1 also displayed low levels of labeling (~10%) at the expected adduct mass, as well an unidentified smaller adduct (139 Da less). At this point it was not clear whether the peptides that did not label 14-3-3σ failed to do so due to diminished noncovalent binding affinity or due to suboptimal positioning of the electrophile within the noncovalent complex. Therefore, we exploited the fact that the formation of the covalent bond is slow and takes place over a time scale of hours, and performed a fluorescence polarization binding experiment using a BDP-labeled peptide derived from YAP-1 that binds 14-3-3σ with an affinity of ~100 nM. 14-3-3σ was added at a concentration of 0.25 μM to premixed fluorescent peptide (5 nM) and electrophilic peptides (5 μM or 200 μM) and measured the fluorescence polarization at 27° C. immediately following the mixture (FIG. 9). Only peptides SEQ ID 10 and SEQ ID 11 had sufficient affinity to displace the fluorescent binder at 5 μM, while all peptides except peptide SEQ ID 2 displaced the binder at 200 μM, which is the concentration at which the initial screen was performed. Therefore, while many of the electrophilic peptides have diminished noncovalent affinity to 14-3-3σ, it is likely that the positioning of the electrophile in the noncovalent complex also plays an important role in covalent bond formation. These peptides were analyzed further using time course labeling experiments at lower peptide concentrations (5 μM) at 25° C. Peptides SEQ ID 3 and SEQ ID 8 reached 60% and 80% labeling within 5 hours, respectively. Incubation at 37° C. increased the rate of labeling roughly 4-fold (FIG. 10). Interestingly, when incubated with SEQ ID 11, non-labelled 14-3-3σ disappeared rapidly—within 2.5 hours less than 5% free 14-3-3σ remained. However, the reaction initially yielded a mixture of full peptide-labeled protein (+1275 Da) and protein modified with only the methacrylate group (+112 Da). The methacrylate-labeled protein was gradually converted to full peptide labeled protein (FIG. 3B).

The intrinsic reactivity and stability of the active peptides with their labeling rates were compared). Their stability was tested in buffer and in the presence of lysine and cysteine at room temperature. The peptides showed good stability in buffer—In a timescale of 6 hours in buffer, all peptides were more than >90% intact. Over a timescale of days, peptides SEQ ID 8 and SEQ ID 11 formed a new product with the same mass as the original peptide, possibly due to an internal reaction within the peptide. Peptide SEQ ID 3 remained unmodified even after 3 days. Incubation with lysine did not result in any products other than those observed after prolonged incubation in buffer, indicating the peptides had low intrinsic reactivity towards lysine. The reactivity towards cysteine was much higher—the peptides reacted with cysteine to generate both Michael adducts as well as substitution products in which the thiol peptide is released. Peptides SEQ ID 3, SEQ ID 8 and SEQ ID 11 were more than >50% reacted within 2.5 hours.

### Methods:

### Preparation of Recombinant 14-3-3σ

A pPROEX HTb expression vector encoding the human 14-3-3σ with an N-terminal His6-tag was transformed by heat shock into NiCo21 (DE3) competent cells. Single colonies were cultured in 50 mL LB medium (100 mg/ml ampicillin). After overnight incubation at 37° C., cultures were transferred to 2 L TB media (100 mg/ml ampicillin, 1 mM MgCl₂) and incubated at 37° C. until an OD600 nm of 0.8-1.2 was reached. Protein expression was then induced with 0.4 mM isopropyl-β-d-thiogalactoside (IPTG), and cultures were incubated overnight at 18° C. Cells were harvested by centrifugation (8600 rpm, 20 minutes, 4° C.) and resuspended in lysis buffer (50 mMHEPES, pH 8.0, 300 mM NaCl, 12.5 mM imidazole, 5 mM MgCl2, 2 mM PME) containing cOmplete™ EDTA-free Protease Inhibitor Cocktail tablets (1 tablet/100 mL lysate) and benzonase (1 μl/100 mL). After lysis using a C3 Emulsiflex-C3 homogenizer (Avestin), the cell lysate was cleared by centrifugation (20 000 rpm, 30 minutes, 4° C.) and purified using Ni⁺²-affinity chromatography (Ni-NTA superflow cartridges, Qiagen). Typically two 5 mL columns (flow 5 mL/min) were used for a 2 L culture in which the lysate was loaded on the column washed with 10 CV wash buffer (50 mMHEPES, pH 8.0, 300 mM NaCl, 25 mM imidazole, 2 mM PME) and eluted with several fractions (2-4 CV) of elution buffer (50 mM HEPES, pH 8.0, 300 mM NaCl, 250 mM imidazole, 2 mM bME). Fractions containing the 14-3-3σ protein were combined and dialyzed into 25 mM HEPES pH 8.0, 100 mM NaCl, 10 mM MgCl₂, 500 μM Triscarboxyethylphosphine (TCEP). Finally, the protein was concentrated to ~60 mg/ml, analyzed for purity by SDS-PAGE and Q-Tof LC/MS and aliquots flash-frozen for storage at −80° C.

### Peptide Synthesis

Reagents for peptide synthesis were purchased from Chem-Impex. Peptides were synthesized on Rink Amide resin using standard Fmoc chemistry on a 0.025 mmol scale. The resin was swelled for 30 minutes in dichloromethane (DCM), then washed with dimethylformamide (DMF). Fmoc deprotections were carried out using 20% piperidine in DMF (3×3 minutes), and couplings were performed as follows: 4 equivalents of amino acid were mixed with 4 equivalents of HATU (Hexafluorophosphate Azabenzotriazole Tetramethyl Uronium)/HOAT (1-Hydroxy-7-azabenzotriazole) and 8 equivalents of DIPEA (Diisopropylethylamine) in DMF and added to the resin with mixing for 30 minutes. For phosphoserine and propargylglycine, 2 equivalents were used and reaction times were extended to 2 hours. After the last Fmoc deprotection, the peptides were acetylated at the N terminus using acetic anhydride (10 equivalents) and DIPEA (20 equivalents) in DMF for 30 minutes. Finally the resins were washed with DCM, dried in a desiccator, and cleaved using 94% TFA/1% DODT(3,6-dioxa-1,8-octanedithiol)/2.5% TIPS(triisopropylsilane)/2.5% water for 2 hours with tumbling. The cleaved peptides were precipitated in cold diethyl ether: hexane, washed once with ether, dried, dissolved in 50% acetonitrile and lyophilized.

The electrophile was introduced directly to the crude peptides as follows: Crude peptides were dissolved in 100 mM NaPi pH=7.5 at a concentration of 25 mM. Ethyl 2-(bromomethyl)acrylate was dissolved in acetonitrile to 200 mM and 3 equivalents were added to the peptide solution. Reactions were monitored using LCMS and were typically complete within 1-2 hours at room temperature. Reacted peptides were then purified using reverse phase HPLC.

To prepare fluorescently labeled peptides, a residue of propargylglycine was coupled to the peptide at the N-terminus prior to N-terminal acetylation, cleavage and reaction with Ethyl 2-(bromomethyl)acrylate. The pure peptide was then labeled as follows using copper-catalyzed azide-alkyne cycloaddition (CuAAC): 5 ul of 20 mM peptide was mixed with 15 μL of BDP-TMR azide (150 nmol). Water was added to 100 μL and about 50 μL tBuOH was added to dissolve the dye. At this point, CuSO₄:THPTA 100 mM (1 μL), and 200 mM sodium ascorbate (200 nmol, freshly dissolved) were added and the reaction continued for 1 hour and the product was purified using HPLC. The purity of all peptides was confirmed using LCMS.

### LC/MS Instrumentation and Runs

The LC/MS runs for 14-3-3σ were performed on a Waters ACQUITY UPLC class H instrument, in positive ion mode using electrospray ionization. UPLC separation used a C4-BEH column (300 Å, 1.7 μm, 21 mm×100 mm). The column was held at 40° C. and the autosampler at 10° C. Mobile phase A was 0.1% formic acid in water, and mobile phase B was 0.1% formic acid in acetonitrile. The run flow was 0.4 mL/min. The gradient used was 1% B for 2 min, increasing linearly to 80% B for 2.5 min, holding at 80% B for 0.5 min, changing to 20% B in 0.2 min, and holding at 1% for 0.8 min. The MS data were collected on a Waters SQD2 detector with an m/z range of 2-3071.98 at a range of 900-1900 m/z. The desolvation temperature was 500° C. with a flow rate of 800 L/h. The voltages used were 1.00 kV for the capillary and 24 V for the cone. Raw data were processed using openLYNX and deconvoluted using MaxEnt with a range of 28000:34000 Da and a resolution of 1 Da/channel.

The LS/MS runs for peptides were performed using the same instrument with a C18-CSH column (300 Å, 1.7 μm, 21 mm×100 mm) using a gradient starting from 1% B for 1 minute, rising to 95% B in 4.5 minutes, holding at 95% B for 0.75 minutes, then decreasing to 1% B in 0.75 minutes and holding at 1% B for 1 minute. MS data were collected at a range of 80-2500 m/z, using identical conditions for ionization as with the protein.

### Example 2

### Modified Peptides can Target Both Cysteine and Lysine, Controlling 14-3-3 Isoform Selectivity

To elucidate the binding sites of the peptides on 14-3-3σ, trypsin digestion was performed followed by LC/MS/MS. (The peptides and 14-3-3σ were prepared as disclosed in Example 1). Direct identification and quantification of modified peptides proved challenging due to long peptide chain lengths, fragmentation from multiple directions and weak relative signals. To characterize peptide ligation sites, we switched strategy and measured the relative change in the signal of non-modified peptides on 14-3-3σ relative to a DMSO-treated control. Specifically, we looked at the peptides containing or immediately following residues Cys38, Lys49 and Lys122 (FIG. 8D). Peptide SEQ ID 11 specifically reduced the signal of the Cys38-containing peptides, with little effect on the signals for other peptides, indicating specific Cys38 binding. In contrast, peptides containing or following Lys122 were significantly depleted by peptides SEQ ID 3 and SEQ ID 8, albeit not uniformly, while the signals of Lys49 containing peptides were slightly increased. This result pointed to Lys122 as the likely binding site for peptides SEQ ID 3 and SEQ ID 8. The non-uniform reduction in signal for Lys122-containing peptides may be due to incomplete labeling of 14-3-3σ by SEQ ID 3 and SEQ ID 8. The possibility that adducts to lysine are unstable in the presence of reducing agents used before tryptic digest was considered. To test this, the peptides were incubated with excess TCEP (Triscarboxyethylphosphine) or DTT (Dithiothreitol), which caused rapid release of the peptide from the methacrylate within two hours (FIG. 8A). However, under the same conditions the peptide-protein adducts were stable (FIG. 8B). The protein was incubated with fluorescently labeled methacrylate peptides, followed by denaturation in DTT-containing sample buffer and SDS-PAGE. Here as well, reduction did not affect the intensity of the fluorescent band, indicating that after aza-Michael addition, the protein-peptide adduct is stable (FIG. 8C).

To further confirm that Lysine 122 is the target residue for peptides SEQ ID 3 and SEQ ID 8, 14-3-3σ was co-crystallized with both peptides. The crystal structures (FIGS. 4A, 4B) clearly showed the covalent bond formed between the amine of Lys122 with the methacrylate, with Lys49 remaining unmodified. Comparison of the crystal structure with the prediction from CovPepDock indicated the model correctly predicted the binding pose around the phosphate and the N-terminal part of the peptide, but less so for the C-terminal region. More specifically, compared to the prediction, Lys122 adopts a more relaxed conformation, the C-terminal residues are not as tightly packed in the binding groove, and in peptide SEQ ID 8 (FIG. 4A) the C-terminal glutamine could not be modeled due to insufficient density. The measured structures of the covalent complexes were compared with the known structures of the noncovalent complexes. For peptide SEQ ID 8, the C terminal part of the peptide was displaced outwards due to the space occupied by the methacrylate ester moiety. The conformation of the peptide SEQ ID 3 complex was far less affected by covalent binding due to the shorter C terminal part of the peptide. In contrast, both peptides exhibited only a minor effect of covalent binding on the structure of the N terminal region. These results indicate that noncovalent interactions with the C-terminal part of the peptide play a minor role in the binding.

Since Lys122 is highly conserved in all 14-3-3 isoforms (in contrast to cysteine 38 which is unique in 14-3-3σ), peptides SEQ ID 3 and SEQ ID 8 were incubated with other isoforms, together with the Cys38-targeting peptide SEQ ID 12. While peptide SEQ ID 12 labeled only the sigma isoform, peptides SEQ ID 3 and SEQ ID 8 labeled all isoforms with similar efficiencies. Taken together these results conclusively validate that peptides SEQ ID 3 and SEQ ID 8 specifically bind to Lys122 via aza-Michael addition.

### Methods:

### Binding Experiments to 14-3-3σ

Peptide 100× stocks were prepared by dissolving in DMSO+5 mM acetic acid and storing at −80° C. Binding of peptides to 14-3-3σ was performed in 25 mM HEPES pH=7.5, 100 mM NaCl, 10 mM MgCl₂. The protein was diluted to 2 μM in assay buffer, and the diluted protein was added to the peptide stock at 100:1 ratio and incubated in various conditions. For analysis, 24 μl sample was mixed with 6 μl of 2.4% formic acid in water and then 10 μl were injected to intact protein LCMS.

### Fluorescence Polarization Experiments:

Fluorescence polarization experiments were performed using TECAN plate reader in dark 384-well plates in volumes of 50 μl in triplicates. The buffer was HEPES 25 mM pH=7.5, 100 mM NaCl, 10 mM MgCl₂, 0.05% IGEPAL. For each sample, 0.5 μl of 100× stock of the competitor peptide (in DMSO+5 mM acetic acid) was added, followed by 25 μl of 10 nM BDP-labeled non-covalent peptide probe. Finally, 25 μl of 0.5 μM 14-3-3 σ was added to the plate and the plate was mixed. Polarization was measured at 27° C.

### LC/MS/MS Characterization of Labeling Sites of Methacrylate Peptides in 14-3-3σ

14-3-3σ was diluted to 2 μM in HEPES 25 mM pH 7.5, 100 mM NaCl, 10 mM MgCl₂, and incubated with 5 μM peptide in samples of 50 μl. The samples were incubated for 48 hours at room temperature, resulting in ~75% labeling by peptide SEQ ID 3, 90% labeling by peptide SEQ ID 8 and 100% labeling by peptide SEQ ID 11. At this point 50 μl of 10% SDS in HEPES 25 mM pH=7.5 was added and DTT was added to 5 mM, followed by incubation at 65° C. for 45 minutes. This was followed by addition of iodoacetamide to 10 mM and incubation of 40 minutes at room temperature in the dark. The samples were then processed using S-trap (Protify) according to the manufacturer's instructions, followed by desalting using Oasis plate (Waters).

Each sample was dissolved in 50 μl of 3% acetonitrile+0.1% formic acid, and 0.5 μl was injected to the column. Samples were analyzed using EASY-nLC 1200 nano-flow UPLC system, using PepMap RSLC C18 column (2 μm particle size, 100 Å pore size, 75 μm diameter×50 cm length), mounted using an EASY-Spray source onto an Exploris 240 mass spectrometer. uLC/MS-grade solvents were used for all chromatographic steps at 300 n/min. The mobile phase was: (A) H2O+0.1% formic acid and (B) 80% acetonitrile+0.1% formic acid. Peptides were eluted from the column into the mass spectrometer using the following gradient: 1-40% B in 60 min, 40-100% B in 5 min, maintained at 100% for 20 min, 100 to 1% in 10 min, and finally 1% for 5 min. Ionization was achieved using a 1900 V spray voltage with an ion transfer tube temperature of 275° C. Initially, data were acquired in data-dependent acquisition (DDA) mode. MS1 resolution was set to 120,000 (at 200 m/z), a mass range of 375-1650 m/z, normalized AGC of 300%, and the maximum injection time was set to 20 ms. MS2 resolution was set to 15,000, quadrupole isolation 1.4 m/z, normalized AGC of 100%, and maximum injection time of 22 ms, and HCD collision energy at 30%. 3 injections of 0.5 μl were performed for each sample. The DDA data was analyzed using MaxQuant 1.6.3.4. The database contained the sequence of the 14-3-3σ construct used in the study, and contaminants were included.

Methionine oxidation and N terminal acetylation were variable modifications, and carbamidomethyl was a fixed modification in the analysis, with up to 4 modifications per peptide. Digestion was defined as trypsin/P with up to 2 missed cleavages. PSM (peptide spectrum match) FDR (false discovery rate) was defined as 1 and Protein FDR/Site Decoy fraction were defined as 0.01. Second Peptides were enabled and Match between runs was enabled with a Match time window of 0.7 minutes. The data was imported into skyline and precursors from 9 peptides containing or following the residues Cys38, Lys49 and Lys122 were selected for parallel reaction monitoring (PRM). In every acquisition cycle, one full MS spectrum was taken at a range of 350-1000 Da, 300% AGC (automatic gain control) target, maximum injection time 20 ms at a resolution of 120,000. Data for each precursor was measured during a 4-5 minute window around the retention time measured in the DDA run, with Q1 resolution of 2 Da, orbitrap resolution of 15,000, 300% AGC target and maximum injection time of 160 ms. The acquired data was then analyzed in skyline using a spectral library generated from the DDA runs. The 3 most intense product ions were used for quantitation relative to the DMSO control. Data have been deposited to the ProteomeXchange Consortium via the PRIDE partner repository with the dataset identifier PXD044257 and 10.6019/PXD044257.

### Crystallization of 14-3-3σ-Peptide Complexes:

14-3-3s was C-terminally truncated (DC=delta C) after T231 (Threonine 231 in the protein) to enhance crystallization. 14-3-3 and SEQ ID 3/SEQ ID 8 peptides were dissolved in complexation buffer (20 mM HEPES pH 7.5, 100 mM NaCl, 10 mM MgCl₂) and mixed in a 1:2.5 or 1:5 molar stoichiometry (protein:peptide) at a final protein concentration of 10, 11, 12 and 12.5 mg/mL. The complex was set up for sitting-drop crystallization after overnight incubation at 4° C., in a custom crystallization liquor (0.095 M HEPES (pH7.1, 7.3, 7.5, 7.7), 0.19 M CaCl₂, 24-29% (v/v) PEG 400 and 5% (v/v) glycerol). Crystals grew within 5-10 days at 4° C.

Crystals were fished and flash-cooled in liquid nitrogen. X-ray diffraction (XRD) data were collected at the Deutsches Elektronen-Synchrotron (DESY) PETRA III beamline P11, Hamburg, Germany.

Initial processing of datasets was done using CCP4i from the CCP4 suite. First, XIA2/DIALS was run for data indexing and integration, and AIMLESS for scaling. The structures were phased by molecular replacement, using protein data bank (PDB) entry 5N75 as a template, in MOLREP. REFMAC5 was used for initial structure refinement. Correct peptide sequences were modeled in the electron density in Coot. The presence of the covalent interaction of the peptides with Lysine 122 was verified by visual inspection of the Fo-Fc and 2Fo-Fc electron density maps in Coot and build in via AceDRG. Finally, REFMAC5 and Coot were used in alternating cycles for model building and refinement. See Table 2 for data collection and refinement statistics.

TABLE 2
Data collection and refinement statistics for
14-3-3σ bound to 3 (PDB:8C2E) and 8 (8C2F)

| PDB | 8C2E | 8C2F |
|---|---|---|
| Protein | 14-3-3σDc | 14-3-3σDc |
| Peptide | SEQ ID 3 | SEQ ID 8 |
| Beam | DESY p11 | DESY p11 |
| Data Collection | | |
| Wavelength (Å) | 1.03322 | 1.03322 |
| Space Group | C 2 2 21 | C 2 2 21 |
| Cell Dimensions | | |
| a, b, c (Å) | 82.5, 111.7, 63.1 | 82.8, 112.2, 63.0 |
| α, β, γ (°) | 90, 90, 90 | 90, 90, 90 |
| Resolution (Å) | 55.92-2.20 (2.27-2.20) | 56.14-2.30 (2.38-2.30) |
| I/σ(I) | 12.1 (6.6) | 8.2 (5.0) |
| Completeness (%) | 99.7 (99.4) | 99.9 (100) |
| Redundancy | 12.8 (10.4) | 13.1 (13.1) |
| CC_{1/2} | 0.991 (0.895) | 0.980 (0.720) |
| Refinement | | |
| Number of | 15133 | 13362 |
| Reflections | | |
| R_{work}/R_{free} | 0.175/0.221 | 0.201/0.267 |
| Number of Atoms | | |
| Protein | 3516 | 3515 |
| Ligand/Ion | 105/4 | 150/4 |
| Water | 110 | 59 |
| B-factors | | |
| Protein | 18.98 | 17.45 |
| Ligand/ion | 18.01/43.35 | 14.18/36.09 |
| Water | 23.72 | 18.83 |
| R.M.S deviations | | |
| Bond Lengths (Å) | 0.0091 | 0.0072 |
| Bond Angles (°) | 1.515 | 1.397 |
| Ramachandran | | |
| Favored (%) | 98.69 | 97.39 |
| Outliers (%) | 0.00 | 0.87 |

### Example 3

### Modified Peptides Detect 14-3-3 Proteins in Lysates and Extracellular Media with High Sensitivity

In Example 2 it was demonstrated that methacrylate peptides can react with all 14-3-3 isoforms, BODIPY-labeled derivatives of peptides SEQ ID 3, SEQ ID 8 and SEQ ID 12 were prepared and tested if they can function as pan-reactive 14-3-3 probes in cell lysates (FIGS. 5A-5B). The methacrylates SEQ ID 3 and SEQ ID 8 formed two main bands, with the bottom band corresponding to a shifted band of 14-3-3β as found by western blot. The bottom band most likely corresponds to the six 14-3-3 isoforms that have very similar sizes (α/β, ζ/δ, γ, σ, η and θ, all 245-248 AAs), while the top band probably corresponds to the F isoform which is larger (255 AAs). The binding of the peptides to 14-3-3 was highly selective with virtually no other proteins significantly labeled in the lysate. Moreover, the bands for the methacrylate peptides intensify after a long incubation (22 h compared to 1 h) indicating their stability under these conditions. The inventors proceeded to test whether the peptides can detect 14-3-3 proteins in extracellular medium. A549 cells were grown in serum-free medium for either 24 hours or 48 hours, and then filtered the medium, concentrated it and exchanged the buffer. The peptides detected 14-3-3 in the medium with very high selectivity and sensitivity. In contrast to the results observed in lysates, 14-3-3σ was not detected by peptide SEQ ID 12 in the medium. Therefore, methacrylate peptides SEQ ID 3 and SEQ ID 8 are powerful tools for the detection and quantitation of 14-3-3 isoforms in lysates and extracellular media.

### Methods:

Measurement of Binding to 14-3-3 Isoforms Using LC-MS qTOF

The 14-3-3 isoforms were buffer-exchanged into complexation buffer (20 mM HEPES pH 7.5, 100 mM NaCl, 10 mM MgCl₂) and mixed with the peptides (3/8) in a 1:5 molar stoichiometry (protein:peptide) at a final concentration of 10 mg/mL. The complexes were buffer-exchanged into milliQ+0.1% formic acid after overnight incubation at 4° C.

UPLC-QToF-MS analysis was performed on a Waters (Milford, MA, USA) Acquity I-Class UPLC system coupled to a Waters Xevo G2 quadrupole time-of-flight (QToF) mass spectrometer. The devices were controlled by MassLynx Software (version 4.1, Waters, MA, USA). Full scan in positive electrospray ionization (ESI+) mode was used as MS acquisition mode with an acquisition range from 200-2000 m/z. A 3 μm, 150×2.0 mm Polaris 3 C8-A column (Agilent, Middelburg, the Netherlands was placed inside a column oven at 60° C. and used for chromatographic separation. Flowrate was set at 0.3 mL/min, and a gradient of water containing 0.1% (v/v) formic acid (A) and acetonitrile containing 0.1% (v/v) formic acid (B) was set as follows (all displayed as % v/v): 0.0-7.5 min (15% to 75% B), 7.5-8.0 min (75% B), 8.0-8.1 min (75% to 15% B), 8.1-10.0 min (15% B). Mass Spectrometry settings were set as follows: capillary voltage: 0.80 kV, cone voltage: 40 V, source offset: 80 V, source temperature: 100° C., desolvation temperature: 400° C., cone gas: 10 L/h desolvation gas: 800 L/h. The samples concentration were 0.01-0.1 mg/mL, and the injection volume was 1 μL. Deconvolution was performed by the MaxEnt1 option of the MassLynx software. Errors were calculated using the MaxEnt Errors option.

### Binding of 14-3-3 Proteins to Peptides in Extracellular Media and Lysates

For experiments in lysates, A549 cells were grown in DMEM+FBS.

The cells were washed with PBS, scraped from the plate and centrifuged 200 g for 5 minutes. The cells were lysed in HEPES 20 mM pH=7.5, 10 mM MgCl₂, 100 mM NaCl with the addition of a protease inhibitor cocktail (Roche 11836170001). Cells were sonicated with 10 pulses of 2 seconds at 22% amplitude using a microprobe, followed by centrifugation at 21000 g for 10 minutes at 4° C. The protein concentration was estimated using BCA, and the lysate was diluted to 1.97 in the lysis buffer.

For incubation with the peptides, 38 μl of either lysate or medium was mixed with 2 μl of 20× stock of the peptide (for peptide SEQ ID 8 and peptide SEQ ID 3:20 μM in 20% DMSO/buffer; for peptide SEQ ID 12: 5 μM in 20% DMSO/lysis buffer; for no peptide: 20% DMSO/lysis buffer), and incubated at 25° C. in the dark. Then, 13.3 μl of 4×LDS (Lithium dodecyl sulfate) samples buffer with 20 mM DTT was added and the samples were heated for 10 minutes at 70° C. The samples were loaded on Bis-Tris gradient gels (4-20%, Genscript) and run using Tris-MOPS buffer at 55 mA/200 V. The gels were transferred to nitrocellulose membrane and the membrane was blocked with 5% BSA/TBST (Bovine serum albumin/Tris buffer saline+Tween20) for 1 hour RT. The membrane was incubated overnight at 4° C. with 1:500 diluted anti-14-3-3β antibody (abcam ab15260) in 5% BSA/TBST. The membrane was washed thrice with TBST and incubated with 1:2000 diluted anti-rabbit Horseradish Peroxidase (HRP) antibody (CST 7074S) for 1 hour RT in 5% BSA/TBST. The membrane was washed thrice with TBST and imaged as followed: fluorescence using 546 nm excitation was measured using ChemiDoc (BioRad) using 9 second exposure, and chemiluminescence was measured using 20 second exposure. Images were processed and generated using ImageLab.

For experiments in medium, after growing the cells they were transferred to DMEM without FBS, followed by incubation for either 24 hours or 48 hours. After the incubation, 8.5 ml of the medium was filtered through a 0.2 μm filter, concentrated using a centrifugal concentrator (vivaspin, cutoff 8000-10000 Da) to ~200 μl, and diluted to 8 ml using HEPES 50 mM pH=7.5, 150 mM NaCl. This was followed by 2 additional rounds of dilution and concentration to ~200 μl. The samples were then diluted to 300 μl with IGEPAL added to 1% as well as protease inhibitors and PhosStop. 38 μl samples from the medium were then incubated with the peptides and analyzed as performed for the lysate, with 15 second exposure for fluorescence and 50 second exposure for chemiluminescence.

For experiments in which the gel was directly imaged, after the run the gel was immersed in fixation solution (45% methanol, 45% water and 10% acetic acid) for 10 minutes, followed by 2 washes with Tris 100 mM pH=8 in water. Afterwards the gel was directly imaged in ChemiDoc.

### Pull Down Proteomics Experiment

N terminally biotinylated derivatives of peptides SEQ ID 3 and SEQ ID 8 were synthesized and purified. A549 cells were harvested and lysed as described before. The lysates were diluted to 1.9 mg/ml in lysis buffer, and the peptides were diluted to 20 μM in 20% DMSO/lysis buffer. 142.5 μl of lysate were mixed with 7.5 μl of 20 μM peptide stock and the samples were incubated at 25° C. for 22 hours. The proteins were precipitated by addition of 450 μl water, 600 μl HPLC grade methanol and 150 μl HPLC grade chloroform, followed by vortexing and centrifugation for 10 minutes at 21000×g at 4° C. The top layer was aspirated, and 600 μl methanol was added and the sample was vortexed and centrifuged again, followed by aspiration of the supernatant. The pellet was air dried and kept at −80° C. The pellet was dissolved in 200 μl of 2.5% SDS in PBS with heating to 60° C. and shaking at 1150 rpm for 30 minutes. After dissolution, the sample was diluted 20-fold in PBS and incubated with 10 μl of streptavidin agarose beads (Thermo) with tumbling for three hours at room temperature.

The beads were then filtered through spin columns in a vacuum manifold. The beads were washed twice with 1% SDS/PBS (300 μl), dispersed in 300 μl 1% SDS/PBS and 3 μl of 1 M DTT were added with 30 minutes incubation at room temperature. Then, 15 μl of freshly dissolved 0.8 M iodoacetamide were added followed by 30 minutes incubation at room temperature in the dark. The solution was then removed and the beads were washed three times with 350 μl of freshly dissolved 6 M urea in PBS, three times with 400 μl of 20% methanol in PBS, once with PBS and twice with water. The beads were then transferred to tubes using 100 μl of 50 mM triethylammonium bicarbonate, and the bound proteins were digested with 0.5 μg of trypsin (promega) at 37° C. with shaking at 1200 rpm for 6 hours.

The beads were centrifuged and the supernatant was mixed 1:1 with 0.2% TFA in water. The peptides were desalted using Oasis desalting columns (Waters) and dried under vacuum. The dry peptides were dissolved in 3% acetonitrile+0.1% formic acid (25 μl) and 2 μl were injected. Samples were analyzed using EASY-nLC 1200 nano-flow UPLC system, using PepMap RSLC C18 column (2 μm particle size, 100 Å pore size, 75 μm diameter×50 cm length), mounted using an EASY-Spray source onto an Exploris 240 mass spectrometer. uLC/MS-grade solvents were used for all chromatographic steps at 300 nL/min. The mobile phase was: (A) H2O+0.1% formic acid and (B) 80% acetonitrile+0.1% formic acid. Peptides were eluted from the column into the mass spectrometer using the following gradient: 1-40% B in 160 min, 40-100% B in 5 min, maintained at 100% for 20 min, 100 to 1% in 10 min, and finally 1% for 5 min. Ionization was achieved using a 2100 V spray voltage with an ion transfer tube temperature of 275° C. Initially, data were acquired in data-dependent acquisition (DDA) mode. MS1 resolution was set to 120,000 (at 200 m/z), a mass range of 375-1650 m/z, normalized AGC of 300%, and the maximum injection time was set to 20 ms. MS2 resolution was set to 15,000, quadrupole isolation 1.4 m/z, normalized AGC of 50%, automatic maximum injection time, and HCD collision energy at 30%. 4 samples were analyzed per condition.

Data analysis was performed using Fragpipe (version 19.1) using Msfragger search engine (version 3.8), IonQuant 1.8.10 and Philosopher 4.8.1. Analysis was performed using a human proteome database from December 2022 (Uniprot) with contaminants added and with Streptavidin added manually as a contaminant. Msfragger analysis was performed using Trypsin as the enzyme that cuts after Arg and Lys, with up to 2 missed cleavages, peptide length 7-50 and the N terminal methionine removed. N terminal acetylation and methionine oxidation were defined as variable modifications and carbamidomethyl was defined as a fixed modification. False discovery rate of 0.01 was used both at the peptide and the protein level. Label-Free Quantification was performed using IonQuant, with Match Between Runs enabled with a tolerance of 1 minute. After analysis, the combined protein file was analyzed using Perseus. Intensities were converted to Log 2 values, the quadruplicates of each type were grouped, and all proteins for which there were at least 3 valid values in one of the groups were kept in the analysis. Missing values were replaced by imputation from a normal distribution (downshift 1.8, width 0.3), and differences and P-values were calculated using student's t-test. The data have been deposited to the ProteomeXchange Consortium via the PRIDE partner repository with the dataset identifier PXD044294.

### Example 4

### Modified Proteins (Im9) Covalent Binders

A recombinant protein was modified into a covalent binder using 2-(bromomethyl)acrylate. As a model system the bacterial Colicin E9 toxin/anti-toxin system was selected. This system is composed of a highly toxic nuclease (E9) which is bound in the cell by an inhibitory partner termed the immunity protein (Im9). The complex can be excreted and internalized by target cells while displacing Im9, leading to E9-induced toxicity. The affinity of the Im9/E9 complex is very high and is well characterized structurally.

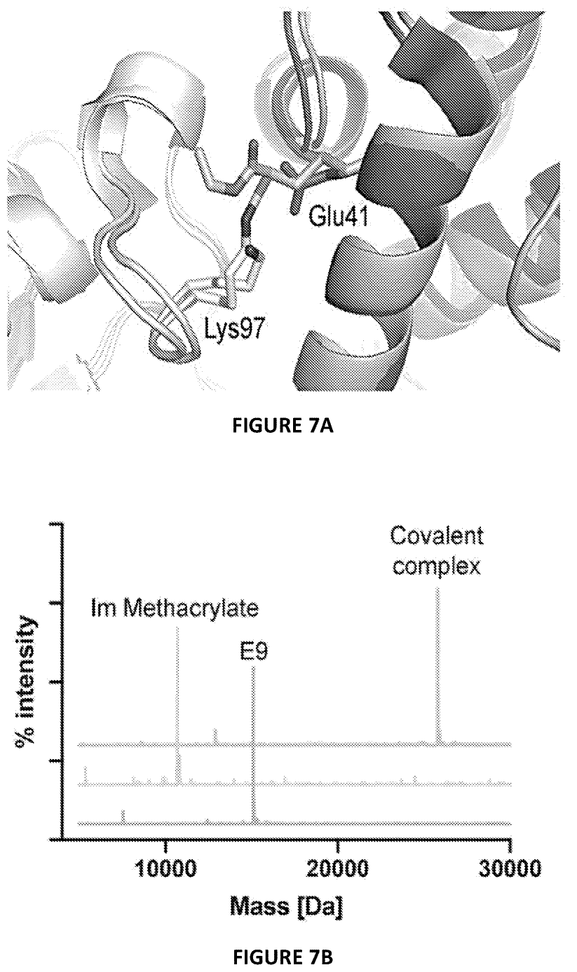

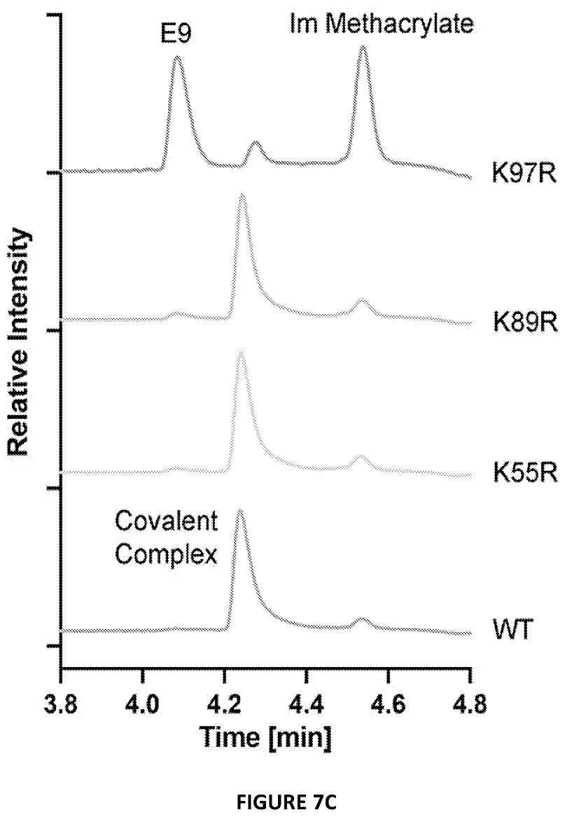

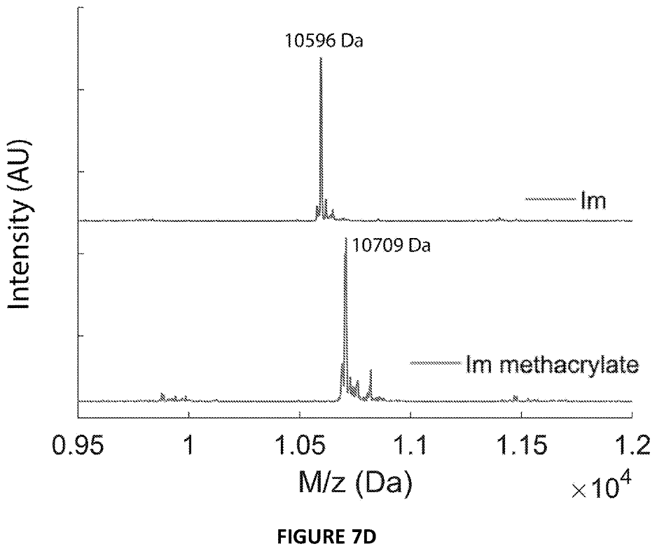

A computational pipeline using Rosetta Relax application (Tyka, M. D. et al., Alternate States of Proteins Revealed by Detailed Energy Landscape Mapping. J. Mol. Biol. 2011, 405 (2), 607-618.; Leman, J. K.; et al., Macromolecular Modeling and Design in Rosetta: Recent Methods and Frameworks. Nat. Methods 2020, 17 (7), 665-680.) was done which performed all-atoms refinement using relatively small moves that sample the local conformational space, while applying covalent constraints between the methacrylate side-chain and the target lysine to enforce the covalent bond between them. Based on the non-covalent complex of colicin E9 and Im9 (PDB: 1EMV), we mutated 20 positions of Im9 that are within Cα-Cα distance<14 Å from the target Lys97 to our methacrylate side-chain, and found five mutations that yielded sub-angstrom models (interface backbone RMSD<1 Å) with particularly good interface and constraint scores 0. From these designs, an Im9 mutant (C23 Å/E41C) was selected onto which a methacrylate ‘warhead’ was installed to react with Lys97 in the E9 nuclease (FIG. 7A). E9 and the Im9 mutants were expressed and purified. Preparation of methacrylate-modified Im9 mutant under native buffer conditions was impractical as modification of the cysteine was slow and was competed by modification of other sites, as observed by the appearance of multiply-labeled species before full formation of the mono-labeled protein was observed, possibly indicating the cysteine was not fully exposed. Preparation under denaturing conditions (50% acetonitrile) was far more efficient, with rapid and selective modification in the time scale of minutes up to an hour, and combined with HPLC purification we obtained >95% single labeled protein (FIG. 7D).

To assess ligation of methacrylate-modified Im9 to Lys97 of E9, modified Im9 mutant and E9 were incubated and the cross linked complex was monitored via intact protein LCMS. The results show about 50% conversion to the covalent complex within 5 hours and near quantitative conversion within 16 hours (FIG. 7B). To validate Lys97 ligation, a point mutation experiment was performed by individually mutating lysines 55, 81, 89, 97 and 125 in E9 to arginine. For the mutants K81R and K125R, bacterial growth was dramatically inhibited, possibly indicating reduction in the binding affinity leading to E9-mediated toxicity. We tested the binding K55R, K89R and K97R to the methacrylate-modified Im9 mutant (FIG. 7C). While the K55R and K89R mutations had no effect on ligation efficiency, mutation of Lys97 abolished the formation of the covalent complex almost completely, indicating Lys97 as the target binding site, in agreement with the model.

The covalent binding affects was studied, by measuring the stability of mutation and covalent binding affects the structure and the complex. To this end, the pure Im proteins and the complexes with E9 were analyzed using SEC-MALS. The estimated Mw of the proteins from the elution volume agree with the MALS measurements and indicated that Im proteins are monomeric, and that the C23 Å/E41C mutation in the Im protein makes the protein adopt a slightly more compact conformation, which may explain the difficulty of modifying the protein in native buffers. The methacrylate-modified mutant behaves more similarly to the WT, and the same trends are observed for the complexes with E9. These results indicate that the covalent complex adopted a similar structure to the native complex. To estimate the effect of the covalent binding on the complex stability, a scanning differential fluorimetry (DSF) was used to monitor the thermal stability of the complex (FIG. 9). Im9 protein in its free and methacrylate-modified form exhibit unfolding around 50° C., while the E9 protein does not show any discernible transition. While the noncovalent complex shows only minimal differences compared to free Im9, the covalent complex is considerably more stable, unfolding at 72° C. (FIG. 9).

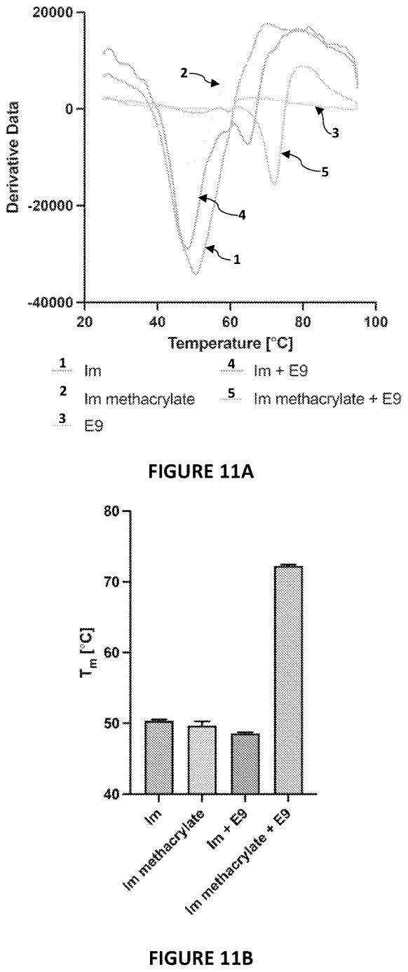

Provided herein a single labeling of Im9 with the methacrylate electrophile (FIGS. 7A-7D). For the purpose of generating covalent protein reagents any number of cysteines can be mutated out, since the protein is generated by standard recombinant expression, supporting single installation of the electrophile. As exemplified herein the electrophilic Im9=irreversibly bind E9 (FIG. 7B). The binding was abrogated, however, by mutation of the target lysine to an arginine (FIG. 7C). Such irreversible protein-protein binding can have significant effects, such as a very strong thermal stabilization as shown for the irreversible Im9/E9 complex (FIGS. 11A-11B) or improved in vivo efficacy. By analogy to peptides, this approach would likely work even better for targeting cysteines on proteins.

### Methods:

### Cloning of Im9 Mutant and E9 Mutants

pET21d plasmids encoding for either E9+wild type Im9 or for Im9 were used. For mutation of E41 intocysteine, PCR was performed using the plasmids as a template using the following primers:

ImFor:
(SEQ ID 13)
GAAATGACTGAGCACCCTAGT
ImRev:
(SEQ ID 14)
ACAAAAGTGTGTAACCAATTTAACCAGTTC

The PCR product was purified and 1 μg was phosphorylated using 10 units T4 PNK (NEB) in 20 μl of T4 ligase buffer (NEB) for 1 hours at room temperature. This was followed by addition of 400 units of T4 ligase (NEB) for 2 hours at room temperature. The product was transformed to DH5α and plated on ampicillin plates. After pick of colonies and identification of correct sequences, this step was repeated with the following primers to introduce the second mutation (C23A):

Im2For:
(SEQ ID 15)
GCTAATGCGGACACTTCCAGTG
Im2Rev:
(SEQ ID 16)
AATTGTTGTTACAAGCTGTAAAAATTCAG

For mutations of E9 we used the same procedure with the following sets of primers:

Lys55for:
(SEQ ID 17)
CGGGCTGTATGGGAAGAGGTGTC
Lys55rev:
(SEQ ID 18)
CCGAAAATCGTCGAAGCTTTTAAATTC
Lys81for:
(SEQ ID 19)
CGAGGTTATTCTCCGTTTACTCCAAAG
Lys81rev:
(SEQ ID 20)
TGAAACACTAGACTTATTGCTTGGG
Lys89for:
(SEQ ID 21)
CGGAATCAACAGGTCGGAGGG
Lys89rev:
(SEQ ID 22)
TGGAGTAAACGGAGAATAACCTTTTG
Lys97for:
(SEQ ID 23)
CGAGTCTATGAACTTCATCATGACAAG
Lys97rev:
(SEQ ID 24)
TCTCCCTCCGACCTGTTG
Lys125for:
(SEQ ID 25)
CGGCGACATATCGATATTCACCG
Lys125rev:
(SEQ ID 26)
AGGTGTAGTCACTCGGATATTATC

### Expression and Purification of E9 and Im9 Mutants

The plasmids were transformed into BL21(DE3) bacteria. The bacteria were grown in 2YT+NPS+1 mM MgSO₄ at 37° C. to OD=0.6, cooled rapidly on ice to 16° C., and induced using 1 mM IPTG for 16 hours.

For purification of Im9, the cells were dispersed in 30 ml of lysis buffer (Tris 25 mM pH=7.5, 50 mM NaCl, 10 mM imidazole)+protease inhibitors, and sonicated (55%, one minute, 5 second pulses). After this, MgCl₂ was added to 1 mM and 5 μl of benzonase nuclease (Fischer) were added. The lysates were spun (20000 rpm for 20 minutes), and the lysates were filtered 0.45 μm. Then, each lysate was loaded on Ni-NTA column (5 ml) preequilibrated with lysis buffer, and the column was washed with 4 CV of lysis buffer. Im9 was eluted with Tris 25 mM pH=7.5, 50 mM NaCl, 500 mM imidazole, dialyzed extensively against NaPi 20 mM pH=7.2, 100 mM NaCl (3 times), filtered 0.2 μm and flash frozen in −80° C.

Purification of E9 was performed using an identical procedure, except that elution was performed using 6 M GuHCl. Some precipitation was observed during dialysis.

### Methacrylate Labeling of Im9 Mutant

Labeling was performed in protein storage buffer (NaPi 20 mM pH=7.2, 100 mM NaCl). Im9(C23 Å/E41C), at a concentration of 1.88 mM, was two-fold in, leading to some precipitation. At his point 1.1 equivalents of ethyl-(3-bromomethacrylate) (dissolved beforehand in acetonitrile) were added. After 1 hour at room temperature, 70% labeling was observed, and another 0.7 equivalents were added. After an hour the sample was diluted in 0.1% TFA in water, filtered and purified using HPLC.

### Reaction Between Im9 Methacrylate and E9

The purified proteins were diluted to 20 μM in NaPi 20 mM pH=7.2, 50 mM NaCl. Then the Im methacrylate solution was mixed in a 1:1 ratio with the E9 solution, giving 10 μM complex. The reactions were incubated at room temperature for 4.5 hours, and then stopped by diluting the complex 5-fold in 0.1% TFA/water, followed by LC-MS analysis.

### Sec-MALS Characterization of Im-E9 Complexes

Samples containing 200 μM isolated Im constructs or Im-E9 complexes were prepared in NaPi 25 mM pH=7.2, 100 mM NaCl. A miniDAWN TREOS multi-angle light scattering detector, with three angles (43.6°, 90° and 136.4°) detectors and a 658.9 nm laser beam, (Wyatt technology, Santa Barbara, CA) with a Wyatt QELS dynamic light scattering module for determination of hydrodynamic radius and an Optilab T-rEX refractometer (Wyatt Technology) were used in-line with size exclusion chromatography analytical Superdex 75 Increase 10/300 GL column (Cytiva). 420-770 μg of each sample were injected to the column in 150-200 μl. Experiments were performed using an AKTA Pure system with a UV-900 detector (Cytiva), at flow rate of 0.8 mL/min and with PBS pH=7.4 as the running buffer. All experiments were performed at room temperature (25° C.). Data collection and SEC-MALS analysis were performed with ASTRA 6.1 software (Wyatt Technology). The refractive index of the solvent was defined as 1.331 and the viscosity was defined as 0.8945 cP (common parameters for PBS buffer at 658.9 nm). dn/dc (refractive index increment) value for all samples was defined as 0.185 mL/g (a standard value for proteins).

### Differential Scanning Fluorimetry for Im-E9 Complexes

50 μl samples of E9:Im complexes in a concentration of 50 μM were prepared and incubated overnight at room temperature in NaPi 20 mM pH=7.2, 50 mM NaCl. SYPRO Orange (X5000 stock) was diluted 200-fold in buffer, and from this stock 13 μl were added to each sample, diluting the protein to 40 μM. Each sample were split into 3 technical replicates and heated in a thermal cycler over 1.5 hours to 95° C. while measuring the fluorescence.

### Introducing New Residues to Rosetta

Our methacrylate side-chain was introduced to Rosetta using the protocol described in Renfrew et al. (Renfrew et al. Incorporation of Noncanonical Amino Acids into Rosetta and Use in Computational Protein-Peptide Interface Design. PLoS One 2012, 7 (3), e32637.) As the reaction between the methacrylate warhead and the lysine amine forms two different stereoisomers, they were implemented as different residues. The GaussView interface was used to draw each stereoisomer, and then used the Gaussian software to optimize the structures, with the following options: HF/6-31G(d) scf=tight test. Each optimized structure was converted to a mol file using OpenBabel toolbox (http://openbabel.org), and then to a Rosetta residue ‘params file’ using the molfile_to_params_polymer.py script provided in Rosetta. To allow the residue to form a covalent bond to another residue, we added a CONNECT record to each stereoisomer params file, specifying which atom participates in the inter-residue covalent bond, as described in Drew et al. (Drew et al. Adding Diverse Noncanonical Backbones to Rosetta: Enabling Peptidomimetic Design. PLoS One 2013, 8 (7), e67051.) for oligooxopiperazines. A virtual atom was added to each params file, and defined its internal coordinates according to the optimal position of the lysine NZ atom as predicted by the Gaussian optimization. These virtual atoms were used during the modeling process to favor the correct covalent bond geometry. Rotamer libraries were generated using the Rosetta MakeRotLib application.

A suitable covalently-linked variant of lysine was implemented through the residue patch system, to utilize the existing definitions and rotamer libraries that have been optimized for use in Rosetta. The reacted lysine was modeled as described above, and created a patch file that deletes the 3 HZ atom of lysine, and adds a CONNECT record and a virtual atom with internal coordinates that match the Gaussian optimized structure. A PROTON_CHI record was added to allow sampling of the new rotamers around the bond CE-NZ bond.

### Design of 14-3-3σ Peptide Binders

PDB ID: 3MHR was used as a template structure to design Lys49- and Lys122-binding peptides for 14-3-3σ. Rosetta fixed backbone design application (fixbb) was used to mutate each lysine to a covalently-linked variant, and the relevant peptide positions (Cα-Cα distance to the target lysine<14 Å) to each of our methacrylate side-chain stereoisomers; these include positions 126-131 for Lys49 and positions 126-133 for Lys 122. The CovPepDock was applied to generate 200 models of each of these mutated complexes (100 for each stereoisomer). To favor the formation of the covalent bond in its correct geometry, AtomPair constraints was applied between each of the covalent bond atoms and its virtual placeholder in the partnering residue, as described in our previous work. HARMONIC score function was used, centered at 0 and with a standard deviation of 0.3. 10 top-interface-scoring models of each complex were manually inspected, focusing on near-native models with constraint score<2, and selected 4 high-ranking peptides.

For the second set of peptides, the PDB for X-ray crystal structures of 14-3-3σ in complex with a 3-15 amino acids long peptide. The results were filtered for structures where the peptide binds near Lys122 (Cα-Cα distance<14 Å) but not near Cys38 (Cα-Cα distance>12 Å). This yielded the PDB IDs 3IQU, 3P1N, 4IEA, 4QLI and 7NWF. Similarly, Lys122-binding peptides were designed for 14-3-3σ based on each of these structures, by mutating positions 257-260 of 3IQU, 372-374 of 3P1N, 620-625 of 4IEA, 175-180 of 4QLI and 592-595 of 7NFW. The native Cys180 of the 4QLI peptide was mutated to serine, to avoid the possible cyclization or side-reactions which may occur due to the addition of the second cysteine onto which we would install the methacrylate warhead.

### Design of Colicin E9 Protein Binders

PDB ID: 1EMV was used as a template structure. Similar to the peptide design protocol, Rosetta fixed backbone design application (fixbb) was used to mutate Lys97 of colicin E9 to our covalently-linked variant, and to mutate positions 30-41 and 48-55 of Im9 to each stereoisomer of our methacrylate side-chain. The RosettaScripts interface was then used and the FastRelax mover to generate 200 models of each complex (100 for each stereoisomer), while applying similar constraints to these described in the peptide design method section. To select a construct for synthesis and testing, the 10 top-interface-scoring models were manually inspected of each mutated complex, focusing on near-native models with constraint score<2.

### Example 5

### Characterization of Selectivity and Off Targets Using Chemical Proteomics

To further characterize the selectivity and off targets of the methacrylate peptides, biotinylated derivatives of peptides SEQ ID 3 and SEQ ID 8 were synthesized and, incubated A549 lysates with them, enriched the biotinylated proteins using streptavidin beads and used trypsin digestion followed by LC-MS/MS to characterize the bound proteins. All isoforms of 14-3-3 were bound efficiently and are the most prominent targets with few off-targets, confirming that peptides SEQ ID 3 and SEQ ID 8 were selective, pan-14-3-3 reactive probes (FIG. 6). Several off-targets were identified, many of which are NAD/NADP dependent enzymes such as aldo-ketoreductases, aldolases and dehydrogenases. These contain a defined binding pocket for the phosphate containing cofactor with nearby lysine residues. Enzymes with phosphate containing substrates, including several glycolytic enzymes, were also prominent off-targets (FIG. 10). We speculate that the phosphorylated peptides may compete for these binding sites and form covalent adducts with these proteins. Nevertheless, the fluorescence imaging results indicated that 14-3-3 proteins were targeted very selectively, and that only a small fraction of the off-targets became modified due to lack of more specific sequence recognition.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A modified protein or a modified peptide comprising a recombinant protein or a synthetic peptide modified by an electrophile, wherein the electrophile is covalently bound to a thiol group of a cysteine within the protein or peptide; wherein the electrophile is represented by the structure of Formula I; 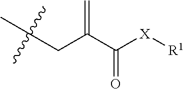
wherein,
R¹ comprises substituted or unsubstituted alkyl, alkenyl, alkynyl, carbocycle, aryl, heteroaryl or heterocyclic;
X comprises O, NR³ or substituted or unsubstituted alkylene; and R³ is hydrogen, substituted or unsubstituted alkyl, alkenyl, alkynyl, carbocycle, aryl, heteroaryl or heterocyclic;
wherein the modified protein or modified peptide is capable of covalently binding a target protein.

2. The modified protein or modified peptide of claim 1, having a non-covalent recognition with the target protein and the electrophile is in close proximity to a target residue of the target protein; prior to the covalent binding with the target protein.

3. The modified protein or modified peptide of claim 2, wherein the target residue within the target protein is a cysteine residue (SH) or a lysine residue (NH₂).

4. The modified protein or modified peptide of claim 2, wherein the close proximity comprises a distance being less than 15 Å between the electrophile and the target residue.

5. The modified protein or modified peptide of claim 1, wherein the electrophile is represented by the structure of Formula II:

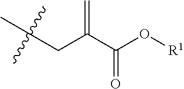

6. The modified protein or modified peptide of claim 1, wherein the electrophile is ethyl methacrylate.

7. The modified peptide of claim 1 comprising the following peptides: Ac-RSApSmCPSL-NH₂ (SEQ ID 3), Ac-RAHpSmCPASLQ-NH₂ (SEQ ID 8) or Ac-RAHpSSPASmCQ-NH₂ (SEQ ID 11); wherein pS is Phoshoserine and mC is methacrylate-modified cysteine.

8. The modified peptide of claim 7, wherein the peptide covalently binds 14-3-3 target protein.

9. The modified peptide of claim 8, wherein the peptide covalently binds Cys38, Lys 122 or Lys 49 of 14-3-3σ target protein.

10. The modified protein of claim 1 comprising a modified immunity (Im9) protein (SEQ ID 27).

11. The modified protein of claim 10, wherein the Im9 protein (SEQ ID 27) binds an E9 target protein.

12. A method of preparing a modified protein or a modified peptide of claim 1, wherein the method comprises;
a. identifying a target protein;
b. designing a peptide or protein covalent binder candidate based on previously characterized noncovalent binders of the target protein;
c. synthesizing a modified peptide or a modified protein, to allow recognition with the target protein at the recognition site; wherein said modified protein or modified peptide comprise a cysteine which is reacted with an electrophile of Formula I.

13. The method of claim 12, wherein the design of the peptide is based on computational modeling comprising Rosetta CovPepDock.

14. The method of claim 12, wherein the modified peptide is synthesized from unprotected peptides.

15. The method of claim 12, wherein the modified protein is synthesized from recombinant proteins.

16. A protein-protein covalent conjugate comprising a modified protein of claim 1, covalently bound to a target protein, wherein the modified protein and the target protein possess non-covalent recognition prior to the covalent binding with the target protein.

17. The protein-protein covalent conjugate of claim 16, wherein the modified protein is Im9 protein and the target protein is E9.

18. The protein-protein covalent conjugate of claim 17, wherein the conjugate displayed a higher thermal stability than the noncovalent complex.

19. A peptide-protein covalent conjugate comprising a modified peptide of claim 1, covalently bound to a target protein, wherein the modified peptide and the target protein possess non-covalent recognition prior to the covalent binding with the target protein.

20. The peptide-protein covalent conjugate of claim 19, wherein the modified peptide is Ac-RSApSmCPSL-NH₂(SEAQ ID 3), Ac-RAHpSmCPASLQ-NH₂ (SEQ ID 8) or Ac-RAHpSSPASmCQ-NH₂ (SEQ ID 11); wherein pS is Phoshoserine and mC is Methacrylate-modified cysteine; and the target protein is 14-3-3.

21. (canceled)


SEQUENCE LISTING (38 sequences)

SEQ ID NO: 1 - protein, 6 aa, synthetic construct
  REGION 1..6: note = Description of sequence: Enzyme: 4QLI_4
  MOD_RES 3: note = T at position 3 is Phosphothreonine
  MOD_RES 4: note = C at position 4 is Methacrylate-modified cysteine
SHTCPS

SEQ ID NO: 2 - protein, 6 aa, synthetic construct
  REGION 1..6: note = Description of sequence: Enzyme: 4QLI_5
  MOD_RES 3: note = T at position 3 is Phosphothreonine
  MOD_RES 5: note = C at position 5 is Methacrylate-modified cysteine
SHTLCS

SEQ ID NO: 3 - protein, 8 aa, synthetic construct
  REGION 1..8: note = Description of sequence: Enzyme: 4IEA_5
  MOD_RES 4: note = S at position 4 is Phosphoserine
  MOD_RES 5: note = C at position 5 is Methacrylate-modified cysteine
RSASCPSL

SEQ ID NO: 4 - protein, 8 aa, synthetic construct
  REGION 1..8: note = Description of sequence: Enzyme: 4IEA_7
  MOD_RES 4: note = S at position 4 is Phosphoserine
  MOD_RES 7: note = C at position 7 is Methacrylate-modified cysteine
RSASEPCL

SEQ ID NO: 5 - protein, 8 aa, synthetic construct
  REGION 1..8: note = Description of sequence: Enzyme: 4IEA_8
  MOD_RES 4: note = S at position 4 is Phosphoserine
  MOD_RES 8: note = C at position 8 is Methacrylate-modified cysteine
RSASEPSC

SEQ ID NO: 6 - protein, 6 aa, synthetic construct
  REGION 1..6: note = Description of sequence: Enzyme: 3IQU_6
  MOD_RES 5: note = S at position 5 is Phosphoserine
  MOD_RES 6: note = C at position 6 is Methacrylate-modified cysteine
QRSTSC

SEQ ID NO: 7 - protein, 6 aa, synthetic construct
  REGION 1..6: note = Description of sequence: Enzyme: 3P1N_6
  MOD_RES 5: note = S at position 5 is Phosphoserine
  MOD_RES 6: note = C at position 6 is Methacrylate-modified cysteine
KRRKSC

SEQ ID NO: 8 - protein, 10 aa, synthetic construct
  REGION 1..10: note = Description of sequence: Enzyme: 3MHR_5
  MOD_RES 4: note = S at position 4 is Phosphoserine
  MOD_RES 5: note = C at position 5 is Methacrylate-modified cysteine
RAHSCPASLQ

SEQ ID NO: 9 - protein, 10 aa, synthetic construct
  REGION 1..10: note = Description of sequence: Enzyme: 3MHR_7
  MOD_RES 4: note = S at position 4 is Phosphoserine
  MOD_RES 7: note = C at position 7 is Methacrylate-modified cysteine
RAHSSPCSLQ

SEQ ID NO: 10 - protein, 10 aa, synthetic construct
  REGION 1..10: note = Description of sequence: Enzyme: 3MHR_8
  MOD_RES 4: note = S at position 4 is Phosphoserine
  MOD_RES 8: note = C at position 8 is Methacrylate-modified cysteine
RAHSSPACLQ

SEQ ID NO: 11 - protein, 10 aa, synthetic construct
  REGION 1..10: note = Description of sequence: Enzyme: 3MHR_9
  MOD_RES 4: note = S at position 4 is Phosphoserine
  MOD_RES 9: note = C at position 9 is Methacrylate-modified cysteine
RAHSSPASCQ

SEQ ID NO: 12 - protein, 10 aa, synthetic construct
  REGION 1..10: note = Description of sequence: Enzyme: 3MHR_CI
  MOD_RES 4: note = S at position 4 is Phosphoserine
  MOD_RES 10: note = X at position 10 is Gamma-chloroacetamido-diaminobutyric acid
RAHSSPASLX

SEQ ID NO: 13 - DNA, 21 bp, synthetic construct
  source 1..21: mol_type = other DNA
  misc_feature 1..21: note = Description of sequence:ImFor
gaaatgactgagcaccctagt

SEQ ID NO: 14 - DNA, 30 bp, synthetic construct
  source 1..30: mol_type = other DNA
  misc_feature 1..30: note = Description of sequence:ImRev
acaaaagtgtgtaaccaatttaaccagttc

SEQ ID NO: 15 - DNA, 22 bp, synthetic construct
  source 1..22: mol_type = other DNA
  misc_feature 1..22: note = Description of sequence:Im2For
gctaatgcggacacttccagtg

SEQ ID NO: 16 - DNA, 29 bp, synthetic construct
  source 1..29: mol_type = other DNA
  misc_feature 1..29: note = Description of sequence:Im2Rev
aattgttgttacaagctgtaaaaattcag

SEQ ID NO: 17 - DNA, 23 bp, synthetic construct
  source 1..23: mol_type = other DNA
  misc_feature 1..23: note = Description of sequence:Lys55for
cgggctgtatgggaagaggtgtc

SEQ ID NO: 18 - DNA, 27 bp, synthetic construct
  source 1..27: mol_type = other DNA
  misc_feature 1..27: note = Description of sequence:Lys55rev
ccgaaaatcgtcgaagcttttaaattc

SEQ ID NO: 19 - DNA, 27 bp, synthetic construct
  source 1..27: mol_type = other DNA
  misc_feature 1..27: note = Description of sequence:Lys81for
cgaggttattctccgtttactccaaag

SEQ ID NO: 20 - DNA, 25 bp, synthetic construct
  source 1..25: mol_type = other DNA
  misc_feature 1..25: note = Description of sequence:Lys81rev
tgaaacactagacttattgcttggg

SEQ ID NO: 21 - DNA, 21 bp, synthetic construct
  source 1..21: mol_type = other DNA
  misc_feature 1..21: note = Description of sequence:Lys89for
cggaatcaacaggtcggaggg

SEQ ID NO: 22 - DNA, 26 bp, synthetic construct
  source 1..26: mol_type = other DNA
  misc_feature 1..26: note = Description of sequence:Lys89rev
tggagtaaacggagaataaccttttg

SEQ ID NO: 23 - DNA, 27 bp, synthetic construct
  source 1..27: mol_type = other DNA
  misc_feature 1..27: note = Description of sequence:Lys97for
cgagtctatgaacttcatcatgacaag

SEQ ID NO: 24 - DNA, 18 bp, synthetic construct
  source 1..18: mol_type = other DNA
  misc_feature 1..18: note = Description of sequence:Lys97rev
tctccctccgacctgttg

SEQ ID NO: 25 - DNA, 23 bp, synthetic construct
  source 1..23: mol_type = other DNA
  misc_feature 1..23: note = Description of sequence:Lys125for
cggcgacatatcgatattcaccg

SEQ ID NO: 26 - DNA, 24 bp, synthetic construct
  source 1..24: mol_type = other DNA
  misc_feature 1..24: note = Description of sequence:Lys125rev
aggtgtagtcactcggatattatc

SEQ ID NO: 27 - protein, 94 aa, synthetic construct
  REGION 1..94: note = Description of sequence: Enzyme: modified Im9 mutant
  MOD_RES 41: note = C at position 41 is Methacrylate-modified cysteine
MELKHSISDYTEAEFLQLVTTIANADTSSEEELVKLVTHFCEMTEHPSGSDLIYYPKEGDDDSPSGIVNTVKQWRAANGKSGFKQGLEHHHHHH

SEQ ID NO: 28 - protein, 275 aa, synthetic construct
  REGION 1..275: note = his-tagged 14-3-3 sigma
SYYHHHHHHDYDIPTTENLYFQGAMGSMERASLIQKAKLAEQAERYEDMAAFMKGAVEKGEELSCEERNLLSVAYKNVVGGQRAAWRVLSSIEQKSNEEGSEEKGPEVREYREKVETELQGVCDTVLGLLDSHLIKEAGDAESRVFYLKMKGDYYRYLAEVATGDDKKRIIDSARSAYQEAMDISKKEMPPTNPIRLGLALNFSVFHYEIANSPEEAISLAKTTFDEAMADLHTLSEDSYKDSTLIMQLLRDNLTLWTADNAGEEGGEAPQEPQS

SEQ ID NO: 29 - protein, 14 aa, synthetic construct
GAVEKGEELSCEER

SEQ ID NO: 30 - protein, 9 aa, synthetic construct
GEELSCEER

SEQ ID NO: 31 - protein, 8 aa, synthetic construct
NLLSVAYK

SEQ ID NO: 32 - protein, 15 aa, synthetic construct
NLLSVAYKNVVGGQR

SEQ ID NO: 33 - protein, 7 aa, synthetic construct
VFYLKMK

SEQ ID NO: 34 - protein, 12 aa, synthetic construct
VFYLKMKGDYYR

SEQ ID NO: 35 - protein, 7 aa, synthetic construct
MKGDYYR

SEQ ID NO: 36 - protein, 18 aa, synthetic construct
MKGDYYRYLAEVATGDDK

SEQ ID NO: 37 - protein, 16 aa, synthetic construct
GDYYRYLAEVATGDDK

SEQ ID NO: 38 - protein, 10 aa, synthetic construct
  MOD_RES 1: note = X at position 1 is BODIPY propargylglycine
  MOD_RES 5: note = S at position 5 is Phosphoserine
  MOD_RES 10: note = X at position 10 is diaminobutyric acid
XRAHSPASLX